(19) United States
(12) Patent Application Publication
(10) Pub. No.: US 2019/0320654 A1
(43) Pub. Date: Oct. 24, 2019

(54) VOLATILE ORGANIC COMPOUNDS FOR INHIBITING FUNGAL GROWTH

(71) Applicant: Georgia State University Research Foundation, Inc., Atlanta, GA (US)

(72) Inventors: Kyle T. Gabriel, Atlanta, GA (US); Christopher T. Cornelison, Atlanta, GA (US)

(21) Appl. No.: 16/460,349

(22) Filed: Jul. 2, 2019

Related U.S. Application Data
(62) Division of application No. 15/502,698, filed on Feb. 8, 2017, now abandoned.
(60) Provisional application No. 62/036,497, filed on Aug. 12, 2014.
(60) Provisional application No. 62/047,433, filed on Sep. 8, 2014.

(51) Int. Cl. A01N 35/02; A61K 31/426; A01N 27/00; A61K 31/10; A61K 31/11; A61K 31/131; A01N 25/06; A01N 43/78; A61K 9/12; A61D 7/00; A01N 43/72; A01N 37/02; A01N 35/04; A01N 33/04; A01N 31/14; A01N 31/04; A01N 31/02; A61K 31/045

(52) U.S. Cl. CPC A01N 35/02; A61K 31/045; A01N 27/00; A61K 31/10; A61K 31/11; A61K 31/131; A01N 25/06; A01N 43/78; A61K 9/12; A61D 7/00; A01N 43/72; A01N 37/02; A01N 35/04; A01N 33/04; A01N 31/14; A01N 31/04; A01N 31/02; A61K 31/426

(57) ABSTRACT

Compositions, devices, and methods are disclosed for treating or preventing fungal infection in an animal are provided. The methods involve exposing the animal to one or more volatile organic compounds (VOCs) in a quantity sufficient to inhibit or reduce fungal growth in the animal. Also disclosed is an automated aerosolization unit (AAU) for delivering compositions, such as the disclosed VOCs, to areas, such as habitats, to treat or prevent fungal infections in animals.

### CROSS-REFERENCE TO RELATED APPLICATIONS

This application is a divisional of copending application Ser. No. 15/502,698, filed Feb. 8, 2017, which is the National Stage of International Application No. PCT/US2015/044807, filed Aug. 12, 2015, which claims benefit of and priority to U.S. Provisional Patent Application Ser. No. 62/036,497, filed Aug. 12, 2014, and U.S. Provisional Patent Application Ser. No. 62/047,433, filed Sep. 8, 2014, all of which are fully incorporated by reference.

### BACKGROUND

White-nose syndrome (WNS) was first documented near Albany, N.Y., in 2006 [Blehert D S, et al. Science. 2008 323:227; Gargas A, et al. Mycotaxon. 2009 108:147-54]. Since its discovery, WINS has caused severe declines in bat populations in the Eastern United States and Canada [Frick W F, et al. Science. 2010 329:679-82; Turner G G, et al. Bat Res News. 2011 52:13-27]. Although the exact ecological and economic impact of this disease has yet to be determined, many researchers agree that continued declines in insectivorous bat populations will have a significant impact on forest management, agriculture and insect-borne disease [Boyles J G, et al. Science. 2011 332:41-2]. The rapid spread of WNS and the high mortality rates associated with the disease necessitate the rapid development of disease management tools. In 2011, the fungus Geomyces destructans was shown to be the putative causative agent of WNS [Lorch J M, et al. Nature. 2011 480:376-8].

Recently, the fungus Geomyces destructans has been reclassified as Pseudogymnoascus destructans [Lorch J M, et al. Mycologia. 2013 105:237-52; Minnis A M, et al. Fungal Biol. 2013]. P. destructans is a psychrophilic ascomycete with optimal growth at 12.5-15.8° C. [Gargas A, et al. Mycotaxon. 2009 108:147-54; Turner G G, et al. Bat Res News. 2011 52:13-27]. Its psychrophilic nature makes P. destructans ideally suited for colonization of bats in torpor, when body temperatures and immune function are greatly depressed [Boyles J G, et al. Front Ecol Environ. 2010 8:92-8; Casadevall A. Fungal Genet Biol. 2005 42:98-106]. The clinical manifestation of P. destructans infection is characterized by fuzzy white growth on the muzzle and wings of hibernating bats and results in severe physical damage to bat wing membranes [Cryan P, et al. BMC Biol. 2010 8:135]. Due to the recent identification of P. destructans, many ecological and physiological traits and their influence on virulence are yet to be elucidated.

### SUMMARY

Compositions, devices, and methods are disclosed for treating or preventing microbial infections, such as fungal or bacterial infections, are provided. The methods involve exposing an animal or location to one or more volatile organic compounds (VOCs) in gaseous form in a quantity sufficient to inhibit or reduce microbial growth in and on the animal or location.

Also disclosed is an automated aerosolization unit (AAU) for delivering gaseous compounds, such as the disclosed VOCs, to remote and difficult to access areas. For example, the AAU can be used to deliver gaseous antimicrobial compounds to animal habitats or feeding areas to treat or prevent microbial infection in the animals. The AAU may also be used to disperse gaseous antimicrobial compounds through HVAC systems, food storage buildings, and industrial machinery. The AAU may also be used to deliver vaccines to subjects topically, e.g., by respiration.

The AAU is comprised of a computing device, a nebulizer unit, and a power source. In some embodiments, the nebulizer unit is comprised of a reservoir, a pneumatic pump unit, and a nebulizer, wherein the computing device executes computer-readable instructions to disperse a liquid contained in the reservoir in aerosol form to reach a desired concentration in an airspace in which the AAU is placed. The nebulizer unit can further be connected with and controlled by a control device. In some embodiments, the power source comprises one or more of a battery, a capacitor, an energy harvesting device, or an AC power source converted into a form acceptable for the computing device and the nebulizer unit. The computing device can in some embodiments further comprise a voltage and a temperature sensor.

In particular embodiments, the liquid to be dispersed in aerosol form comprises antimicrobial compounds, such as essential oils, VOCs, or VOC formulations. For example, the VOCs can be selected from the group consisting of 2-ethyl-1-hexanol, benzaldehyde, benzothiazole, decanal, nonanal, N,N-dimethyloctylamine, propionoic acid, 2-nonanone, undecene, styrene, β-phenylethanol, and dimethyl sulfide. The VOCs can be combined to increase effectiveness. For example, the liquid to be dispersed in aerosol form can comprise 2-ethyl-1-hexanol and benzaldehyde; 2-ethyl-1-hexanol and nonanal; 2-ethyl-1-hexanol and decanal; or 2-ethyl-1-hexanol and N,N-dimethyloctylamine. In some cases, the liquid to be dispersed in aerosol form comprises 2-ethyl-1-hexanol, benzaldehyde, and decanal. In some cases, the liquid to be dispersed in aerosol form comprises 2-ethyl-1-hexanol, nonanal, and decanal.

The computing device can further comprise an input device. For example, the input device can be removable from the computing device. The input device can be used to enter input parameters into the computing device, such as a mode of operation for the AAU. In some embodiments, the input parameters include one or more of an off time for the AAU, a run time for the AAU, a start delay for the AAU, a volume of airspace where the AAU is to be used, an air turnover rate in the airspace, a barometric pressure in the airspace, absorption capacity of the treatment environment, a molecular weight of the liquid in the reservoir, a desired gaseous concentration of the liquid in the airspace, and how often to raise the airspace to the desired gaseous concentration. The computing device in some cases executes computer-readable instructions to determine how long (Run Time) the device should run in order to reach the desired concentration in the airspace. This run time can be determined using at least in part an ideal gas law.

In some embodiments, the AAU can further comprise a clock module to control delayed start, run time and off time of the AAU. In other embodiments, the computing device can execute computer-readable instructions to delay a start of the AAU; turn on the AAU for a time (T=Run Time); apply a conversion factor to the run time, T, to determine TConv, where the conversion factor is based on a temperature and voltage of the power source; and, if TConv is less than run time T, then the AAU is turned on for an additional time period as determined by T−Tconv.

Also disclosed is a method of automatically dispersing a liquid in aerosol form that involves receiving, by a computing device, one or more input parameters; determining, by the computing device based on the input parameters, how long to run a nebulizer unit operably connected with the computing device to achieve a concentration of a compound in an airspace; and running the nebulizer unit for the determined run time. For example, the compound to be dispersed in aerosol form can contain essential oils, VOCs, or VOC formulations.

The input parameters can include one or more of an off time for the nebulizer unit, a run time for the nebulizer unit, a start delay for the nebulizer unit, a volume of airspace where the nebulizer unit is to be used, an air turnover rate in the airspace, a barometric pressure in the airspace, absorption capacity of the treatment environment, a molecular weight of the compound in the reservoir, a desired concentration of the compound in the airspace, and how often to raise the airspace to the desired concentration. The determined run time and off time can be determined using at least in part an ideal gas law. In some embodiments, the computing device executes computer-readable instructions to delay a start of the nebulizer unit; turn on the nebulizer unit for a time (T=Run Time); apply a conversion factor to the run time, T, to determine TConv, where the conversion factor is based on a temperature and voltage of a power source that provides power to the nebulizer unit; and, if TConv is less than run time T, then the nebulizer unit is turned on for an additional time period as determined by T−Tconv. In other embodiments, delayed start, run time and off time can be controlled with a clock module. The disclosed compositions, methods, or AAU can be used to treat or prevent fungal infection in an animal. In some embodiments, the animal is a bat and the fungus is Pseudogymnoascus destructans.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is an exemplary illustration of an embodiment of an automated aerosolization unit (AAU).

FIG. 2A is an exemplary illustration of an embodiment of a nebulizer unit.

FIG. 2B is an alternate embodiment of an AAU from that as shown in FIG. 1.

FIG. 3 is an exemplary illustration of an embodiment of a computing device.

FIG. 4 is a flowchart illustrating functionality of an exemplary software code for an automated aerosolization unit.

FIGS. 5A and 5B are flow diagrams of example operations for providing aerosolization of a liquid using embodiments of an automated aerosolization unit.

FIG. 5C illustrates configurable regimens that the AAU can be programmed to perform in its complex mode.

FIG. 6 is an image of an airspace assay with bacterially produced VOCs.

FIGS. 7A to 7D are graphs showing growth areas of P. destructans mycelial plugs exposed to bacterially produced VOCs at 15° C. at 30 μl (FIG. 7A), 3 μl (FIG. 7B), 0.3 μl (FIG. 7C), respectively in an airspace of a 150 mm×15 mm Petri plate. Growth area of mycelial plugs exposed at 4° C. to 0.3 μl (FIG. 7D) of bacterially produced VOCs. Any of the 6 previously-mentioned VOCs not shown in the legend completely inhibited radial growth for the duration of the experiment.

FIGS. 8A to 8C are graphs showing growth areas of P. destructans mycelial plugs exposed to 2-ethyl-1-hexanol and benzaldehyde (FIG. 8A), 2-ethyl-1-hexanol and decanal (FIG. 8B), or 2-ethyl-1-hexanol and nonanal (FIG. 8C), as well as formulations, at 15° C. Measurements taken every 2 days for 14 days.

FIGS. 9A and 9B are graphs showing growth areas of P. destructans mycelial plugs exposed to 2-ethyl-1-hexanol, benzaldehyde, and decanal (FIG. 9A) or 2-ethyl-1-hexanol, nonanal, and decanal (FIG. 9B), as well as formulations at 15° C. Measurements taken every 2 days for 14 days.

FIG. 10 is a schematic view of an embodiment of an automated aerosolization unit using conventional symbols for electrical components. In this embodiment, the IC (center) is an ATMEGA168-PA-PU microcontroller.

FIG. 11 is a double sided printed circuit board (PCB) layout of an embodiment of an automated aerosolization unit.

FIGS. 12A and 12B are printed circuit boards without (FIG. 12A) and with (FIG. 12B) components in place.

FIG. 13 is a graph showing weight change (left axis, grams) and partial pressure change (right axis, torr) after ethanol nebulization duration (sec). Nebulization durations were from 0.5 to 3 seconds. Circles represent the amount of ethanol nebulized (left axis) and squares represent the partial pressure change that occurred (right axis).

FIG. 14 is a graph showing weight change after formulation or ethanol nebulization duration (sec). Nebulization durations were from 0.5 to 14 seconds. Circles represent the amount of ethanol nebulized and triangles represent the amount of formulation nebulized.

FIG. 15 is a graph showing perceived time (as a percentage of actual time) as voltage changes, while at constant temperatures 5° C. (square), 15° C. (circle), and 30° C. (diamond).

FIG. 16 is a schematic view of an embodiment of an automated aerosolization unit using conventional symbols for electrical components. In this embodiment the IC (labeled 23 I/O 2) is an ATMEGA328P-PU microcontroller.

FIG. 17 is a double sided printed circuit board (PCB) layout of an embodiment of an automated aerosolization unit. The top board is the controller (6.87 cm×4.30 cm) and the bottom is the programmer (6.89 cm×3.29 cm).

FIG. 18 is a flowchart illustrating functionality of an exemplary software code for an automated aerosolization unit.

FIG. 19 is a bar graph showing necropsy results of acutely-exposed and control bats.

FIG. 20 is a bar graph showing necropsy results of chronically-exposed and control bats.

FIG. 21 is a bar graph showing necropsy results of post-hibernation exposed and control bats.

FIG. 22 is a bar graph showing mass loss of samples from the three groups of bats.

FIG. 23 is a schematic view of an embodiment of an automated aerosolization unit using conventional symbols for electrical components.

FIG. 24 is a double sided printed circuit board (PCB) layout of an embodiment of an automated aerosolization unit.

### DETAILED DESCRIPTION

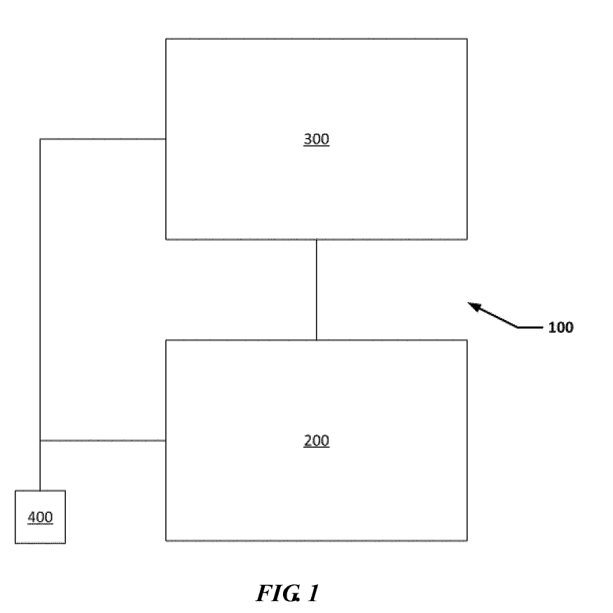

Described herein are embodiments of a device that can distribute VOCs normally produced by microorganisms to inhibit the growth of other microorganisms, but at specific concentrations and in the correct ratio. Development of this device was initiated to combat Pseudogymnoascus destructans (either on a bat or in the environment), the fungal agent responsible for white-nose syndrome in North American Bats, by inhibition of spore germination, inhibition of mycelial growth, inhibition of sporulation, inhibition of pathogenicity, stimulation of immune function, or other potential mechanisms. However, many other potential uses exist, including treatment of other infections, inhibition of microbial growth in difficult-to-access areas, inhibition of microbial growth in areas where liquid antimicrobial treatment is not possible or feasible, among other uses. FIG. 1 is an exemplary illustration of an embodiment of an automated aerosolization unit (AAU) 100 that can be used, for example, to mimic the VOC signature (specific concentrations of VOCs and in the correct ratio) of microorganisms to inhibit the growth of other microorganisms. For example, embodiments of the device shown in FIG. 1 can be used to combat Pseudogymnoascus destructans (either on a bat or in the environment), the fungal agent responsible for white-nose syndrome in North American Bats, by inhibition of spore germination, inhibition of mycelial growth, inhibition of sporulation, inhibition of pathogenicity, stimulation of immune function, or other potential mechanisms. However, many other potential uses exist, including treatment of other infections, inhibition of microbial growth in difficult-to-access areas, inhibition of microbial growth in areas where liquid antimicrobial treatment is not possible or feasible, among other uses. Generally, the AAU 100 utilizes a liquid chemical or chemical formulation that it aerosolizes into, for example, 0.5-5.0 μm diameter droplets, for rapid evaporation into a gaseous state. The AAU 100 can accurately time dispersal intervals to raise an airspace of a known volume to a specific gaseous concentration that is effective at inhibiting microbial growth or pathogenicity, yet below the toxic threshold of inhabitants (if any) in the area being treated (e.g. bats). Generally, there are two main components to the AAU 100, a computing device 300 such as a controller circuit board, and a nebulizer unit 200. For example, the nebulizer unit 200 can be commercially-produced medical nebulizer. Further comprising the AAU 100 shown in FIG. 1 is a power source 400.

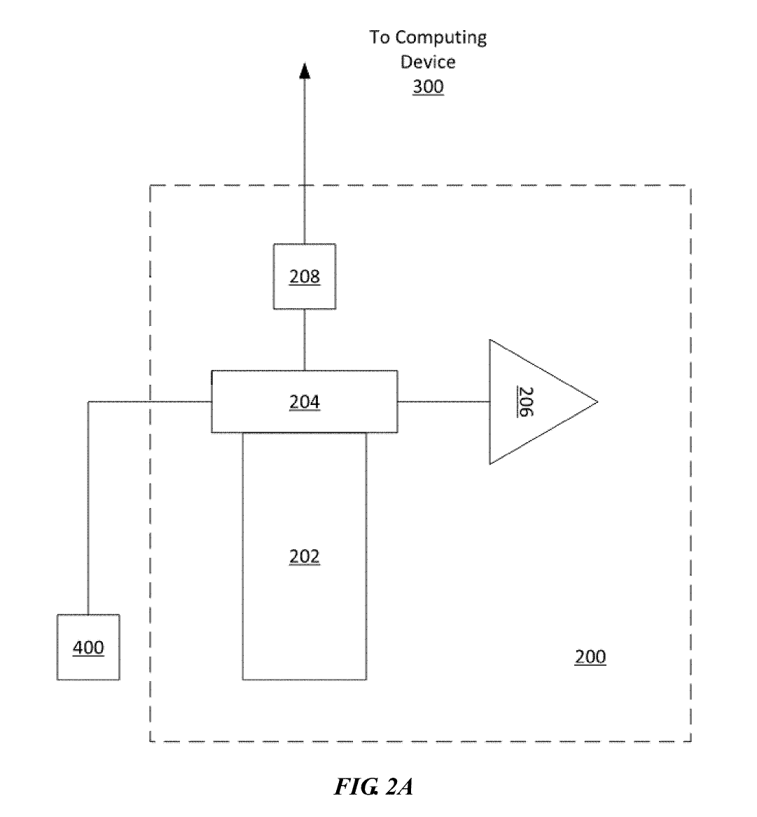

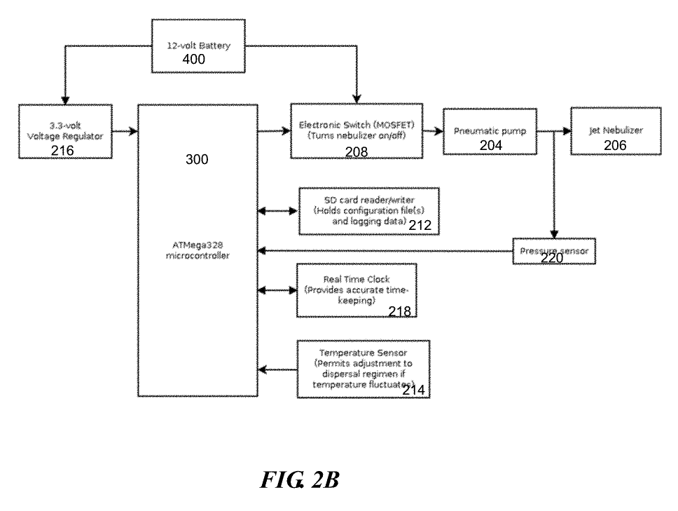

FIG. 2A is an exemplary illustration of an embodiment of a nebulizer unit 200. This embodiment is comprised of a reservoir 202, a pump unit 204, and a nebulizer 206. Optionally, the nebulizer unit 200 may be connected with and controlled by a control device 208, though this device may also be incorporated into or with the computing device 300. For example, the control device 208 can be a MOSFET such as an nMOSFET as available from Fairchild Semiconductor, California. The pump unit 204 is provided power from the power source 400 and controlled by the control device 208 and/or the computing device 300. The reservoir contains the liquid to be dispersed in aerosol form such as, for example, essential oils, and VOCs or VOC formulations, among others. The pump unit 204 and the nebulizer 206 work in concert to transform the liquid in the reservoir 202 to the aerosol form. In one embodiment, the nebulizer unit 206 can be connected through its pump unit 204. The pump unit 204 can be controlled by the control device 208 and/or the computing device 300. Under direction of the control device 208 and/or the computing device 300, the pump unit 204 sends air via an air line to the nebulizer 206. The air goes through the pump unit 204, pulling fluid from the reservoir 202 to the nebulizer 206, where it is aerosolized by the air jet stream. The fluid can comprise a liquid chemical or formulation of chemicals that is loaded into the nebulizer reservoir 202 and the pneumatic pump unit 204 is connected to the nebulizer reservoir 202 to supply pressurized air to the jet within the nebulizer reservoir. Upon pressurizing the jet, an aerosol is formed from the liquid in the reservoir. In some embodiments, the nebulizer reservoir 202 comprises a sensor to monitor fluid levels that is in communication with the control device 208 and/or the computing device 300. In one aspect, the nebulizer unit 200 can be the Pari™ Trek S compressor and LC™ Sprint reusable nebulizer (sold as a set by Pari™, PARI Respiratory Equipment, Inc., Midlothian Va. USA); however other types of nebulizers (ultrasonic, vibrating mesh, etc.) may be connected to the controller and operate similarly. In various embodiments the power source 400 can be a battery, capacitor, energy harvesting device, an AC power source converted into a form acceptable for the computing device 300 and the nebulizer unit 200, and the like.

FIG. 2B illustrates an alternate embodiment of an AAU 100. In this embodiment the computing device 300 comprises a microcontroller (e.g. a microprocessor) that controls the control device 208 (e.g., an electronic switch (MOSFET)) which modulates (turns on and off) the power to the nebulizer compressor 202. The software instructions that the microcontroller runs determines how often the compressor 202 is turned on and off to attain a specific gaseous concentration. This regimen is either explicitly defined by a configuration file on an attached secure digital (SD) memory storage card 212 or is calculated from data acquired from an environmental sensor or sensors 214 (only a temperature sensor is shown in FIG. 2B; however others may be utilized to detect pressure, humidity, etc.). The SD card 212 also permits writing log files, which may include a historical account of temperature, battery voltage, device dispersals, environmental condition changes, and the like. Additionally, because environmental conditions may affect dispersal efficacy, sensors 214 may be used to update the dispersal regimen in order to maintain a consistent gaseous concentration under changing environmental conditions.

In one embodiment, the AAU 100 device design utilizes an ATMega328 microcontroller as the computing device 300 to store the software program and perform calculations. Power may be supplied by, for example, a wall-power 12-volt adapter or a 12-volt battery 400, supplying power to both the controller and the compressor. A voltage regulator 216 can be used to decrease the voltage from, for example, 12 volts to 3.3 volts required by components on the controller circuit board. A MOSFET 208 can be used to switch power on and off to the nebulizer. A real time clock IC (microchip) 218 can be used for accurate time-keeping. A SD card reader/writer 212 can be used to store and read a configuration file from and write logs to a removable memory card. A temperature sensor (thermistor) 214 can be used to retrieve the temperature from the environment.

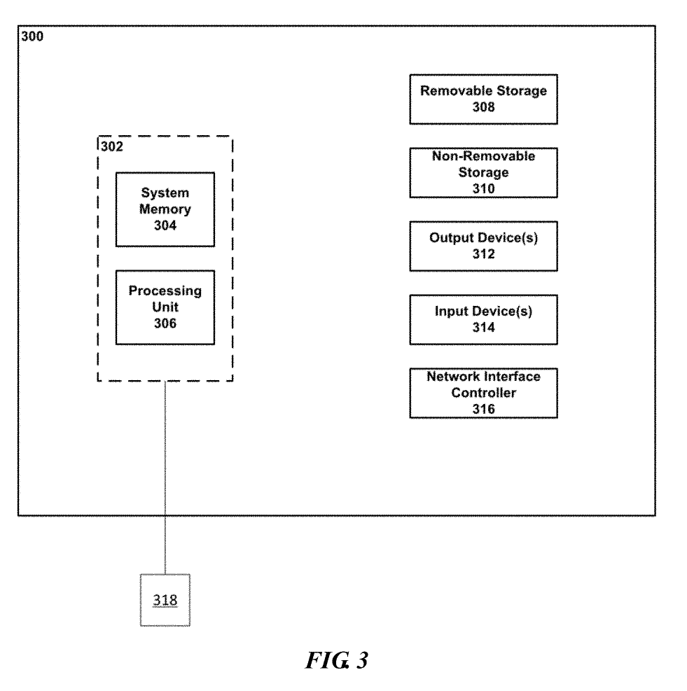

When the logical operations described herein are implemented in software, the process may execute on any type of computing architecture or platform. For example, referring to FIG. 3, an example computing device upon which embodiments of the invention may be implemented is illustrated. In particular, at least one processing device described above may be a computing device, such as computing device 300 shown in FIG. 3. The computing device 300 may include a bus or other communication mechanism for communicating information among various components of the computing device 300. In its most basic configuration, computing device 300 typically includes at least one processing unit 306 and system memory 304. Depending on the exact configuration and type of computing device, system memory 304 may be volatile (such as random access memory (RAM)), non-volatile (such as read-only memory (ROM), flash memory, etc.), or some combination of the two. This most basic configuration is illustrated in FIG. 3 by dashed line 302. The processing unit 306 may be a standard programmable processor that performs arithmetic and logic operations necessary for operation of the computing device 300. For example, the processing unit can be an ATMEGA168-PA-PU microcontroller or an ATMEGA328P-PU microcontroller, as available from Atmel, Calif. These microcontroller chips operate in low-power situations and draw very little current while running and drastically-reduced current usage when in “power-saving” modes. Computing device 300 may have additional features/functionality. For example, computing device 300 may include additional storage such as removable storage 308 and non-removable storage 310 including, but not limited to, magnetic or optical disks or tapes. Computing device 300 may also contain network connection(s) 316 that allow the device to communicate with other devices. Computing device 300 may also have input device(s) 314 such as a keyboard, mouse, touch screen, rotary encoder, etc. In one embodiment, the input device 314 can be connected to the computing device 300 for programming the computing device 300, and then removed after the programming is complete. Output device(s) 312 such as a display, speakers, printer, etc. may also be included. The additional devices may be connected to the bus in order to facilitate communication of data among the components of the computing device 300. All these devices are well known in the art and need not be discussed at length here.

The processing unit 306 may be configured to execute program code encoded in tangible, computer-readable media. For example, the processing unit can be programmed to execute the code shown in Source Code 1, Source Code 2, or Source Code 3 below.

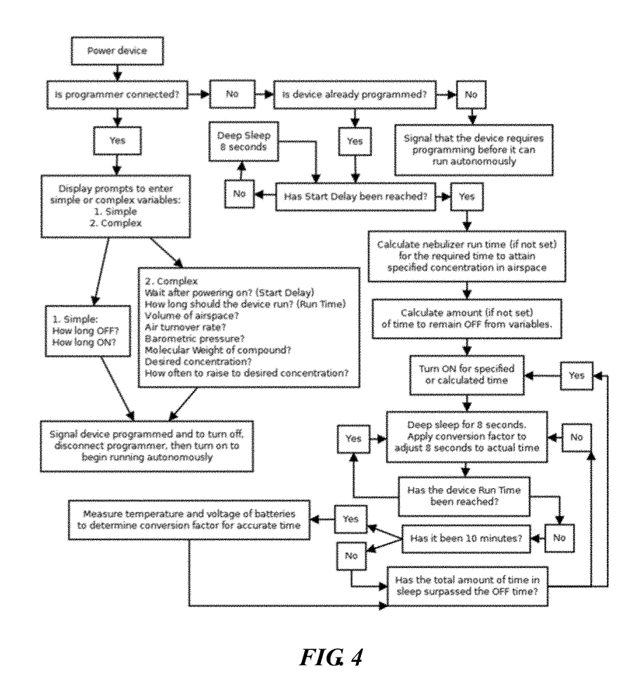

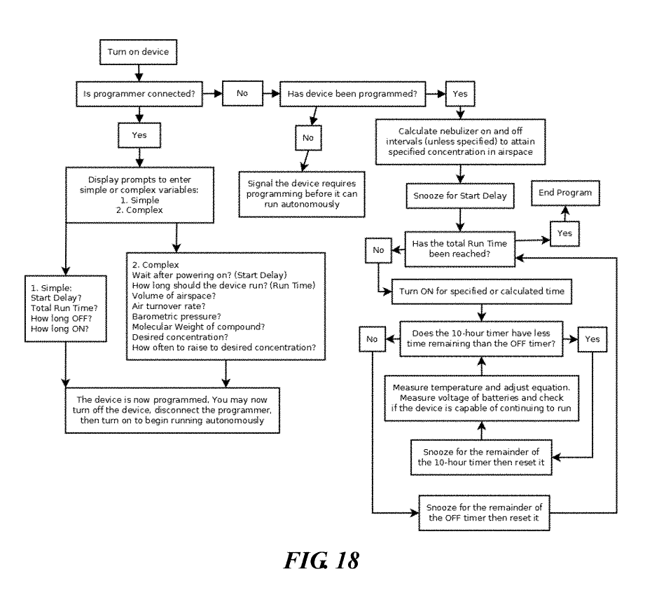

The functionality of the above software code is illustrated in the flowcharts of FIG. 4 (Source Code 1) and FIG. 18 (Source Code 2). It is to be appreciated that the functionality can be programmed in any language or format used or recognizable by the computing device 300 and is not required to be in the form shown above.

Computer-readable media refers to any media that is capable of providing data that causes the computing device 300 (i.e., a machine) to operate in a particular fashion. Various computer-readable media may be utilized to provide instructions to the processing unit 306 for execution. Common forms of computer-readable media include, for example, magnetic media, optical media, physical media, memory chips or cartridges, a carrier wave, or any other medium from which a computer can read. Example computer-readable media may include, but is not limited to, volatile media, non-volatile media and transmission media. Volatile and non-volatile media may be implemented in any method or technology for storage of information such as computer readable instructions, data structures, program modules or other data and common forms are discussed in detail below. Transmission media may include coaxial cables, copper wires and/or fiber optic cables, as well as acoustic or light waves, such as those generated during radio-wave and infra-red data communication. Examples of tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 306 may execute program code stored in the system memory 304. For example, the bus may carry data to the system memory 304, from which the processing unit 306 receives and executes instructions. The data received by the system memory 304 may optionally be stored on the removable storage 308 or the non-removable storage 310 before or after execution by the processing unit 306.

Computing device 300 typically includes a variety of computer-readable media. Computer-readable media can be any available media that can be accessed by device 300 and includes both volatile and non-volatile media, removable and non-removable media. Computer storage media include volatile and non-volatile, and removable and non-removable media implemented in any method or technology for storage of information such as computer readable instructions, data structures, program modules or other data. System memory 304, removable storage 308, and non-removable storage 310 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 300. Any such computer storage media may be part of computing device 300. In one embodiment, the computing device 300 can further comprise voltage and temperature sensors. Temperature and voltage sensing can allow for automatic temperature sensing (for incorporation into ideal gas law calculations) and voltage monitoring (to determine if the batteries can handle the projected run time specified for the AAU). Also, in one embodiment, the computing device 300 can further comprise or be connected with a clock module 318 such as, for example, a DS1337 Real Time Clock module that allows accurate time-keeping and renders having to calculate a conversion factor form voltage and temperature unnecessary. It also has low-power operation and low current draw.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

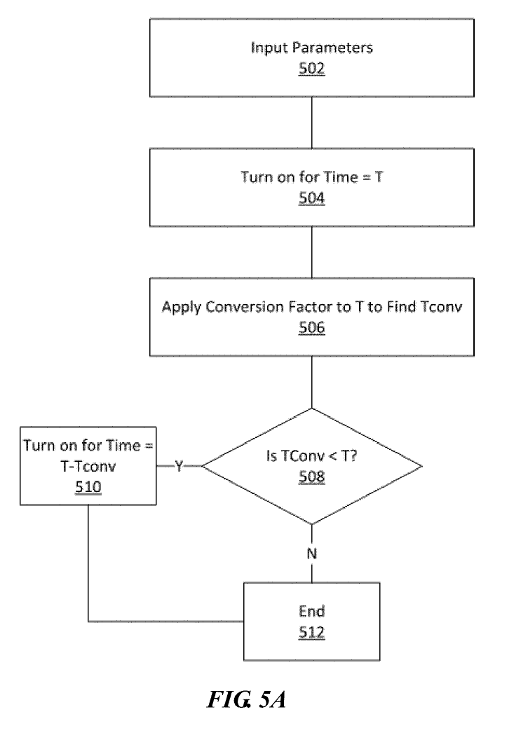

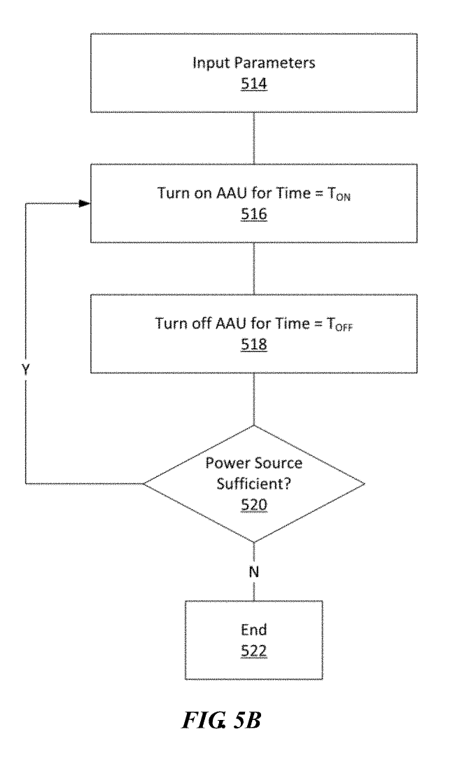

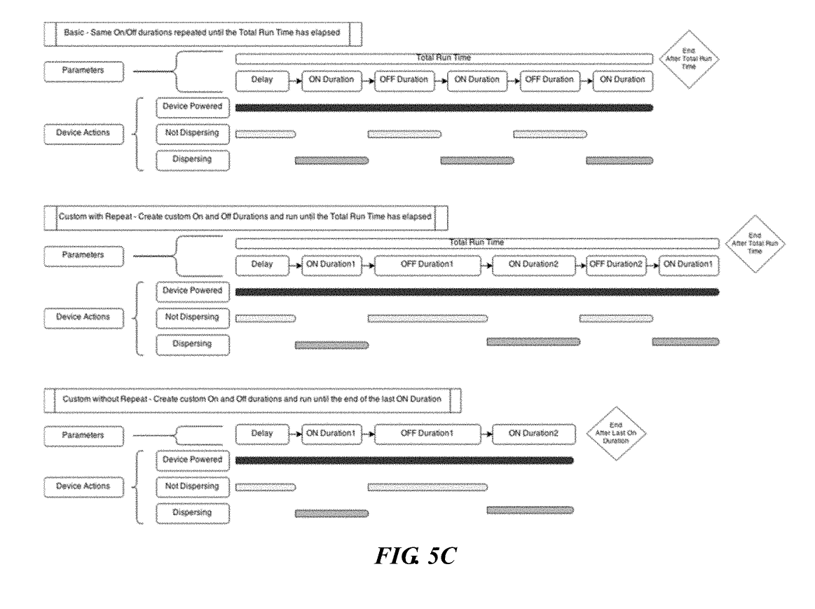

FIGS. 5A and 5B are flow diagrams of example operations for providing aerosolization of a liquid using embodiments of an automated aerosolization unit (AAU) as described herein. Referring to FIG. 5A, at step 502, input parameters are entered into the computing device. For example, the AAU may have different operational modes. One mode can be a simple mode so that the only parameters entered are how long the unit is to be off and how long the unit is to be on. In another more complex mode, parameters can include how long to wait before powering on (Start Delay), how long the device should run (Run Time), the volume of airspace where the device is to be used, the air turnover rate in the airspace, the barometric pressure, the molecular weight of the compound in the reservoir, the desired concentration of the compound in the airspace, and how often to raise the airspace to the desired concentration. Using the ideal gas law, in complex mode, the device can determine how long (Run Time) the device should run in order to reach the desired concentration in the airspace. The Run Time may also be programmed in. At step 504, the AAU turns on for the time (T=Run Time) that is either determined or programmed in step 502. This may occur after a time delay (delay start). After running the designated run time, at step 506 a conversion factor is applied to the run time, T to determine TConv. This conversion factor is based on the temperature and voltage of the power source (e.g. batteries). At step 508, if TConv is less than run time T, then (step 510) the device is turned on for an additional time period as determined by T−Tconv. The process ends at step 512.

FIG. 5B illustrates an alternate flow diagram of example operations for providing aerosolization of a liquid using embodiments of an automated aerosolization unit (AAU) further comprising a clock module 318. Referring to FIG. 5B, at step 514, input parameters are entered into the computing device. For example, the AAU may have different operational modes. One mode can be a simple mode so that the only parameters entered are how long the unit is to be off and how long the unit is to be on. In another more complex mode, parameters can include how long to wait before powering on (Start Delay), how long the device should run (Run Time), the volume of airspace where the device is to be used, the air turnover rate in the airspace, the barometric pressure, the molecular weight of the compound in the reservoir, the desired concentration of the compound in the airspace, temperature, and how often to raise the airspace to the desired concentration, and other environmental conditions. Using the ideal gas law, and monitoring the temperature of the location in which the AAU is placed, in complex mode, the device can determine how long (Run Time) the device should run in order to reach the desired concentration in the airspace. The Run Time may also be programmed into the AAU. At step 516, the AAU turns on for the time (T=ToN) that is either determined or programmed in step 514. This may occur after a time delay (delay start). After running the designated run time, at step 518, the AAU is turned off for a period of time, TOFF. At step 520, the process may then return to step 516, if the power source of the AAU is capable of running for time period ToN, or it may end at step 522. FIG. 5C illustrates configurable regimens that the AAU can be programmed to perform in its complex mode.

In one aspect, multiple AAUs having multiple controllers can be used to coordinate dispersal, providing the ability to utilize different compounds and/or create more complex treatment regimens. This arrangement can provide larger treatment volumes (to a larger air space, for instance) or to create different ratios of a gaseous concentration (such as if each unit has a different VOC or VOC formulation in the reservoir and will coordinate dispersal of each to create a ratio in the air that is different from each unit alone). Each AAU can be equipped with a protective enclosure. All electronics can be housed in a protective case (e.g., Pelican 1150 case, Pelican Products, Inc., Torrance, Calif., USA), which provides protection from moisture/dust intrusion and shock/damage. The enclosure may have an air intake port that with an inline-filter inside the case, that supplies air to the compressor, and an outflow port, which supplies pressurized air to the nebulizer jet.

In one aspect, the AAU can comprises an integrated LED on the controller. The LED allows the user to be visually notified if the device is functioning correctly or if there is an error (for example, Morse code can be used to relay information via the LED). A few uses of the LED include, but are not limited to: indicating that the device has been powered and is currently delaying starting the program (to notify the user the device is powered but is delaying running to allow the user to evacuate the area); indicating that it has been projected, through calculation, that there is not enough power to run the entire program, and a fully-charged battery should be switched with the current battery; or indicating that the device experienced an error while running (consult the error section of a manual to determine the pattern for the particular error).

The microcontroller can be programmed to perform battery power consumption calculations and monitor battery usage. This allows the microcontroller to calculate the projected power consumption of the desired treatment regimen and monitor the battery over the course of the regimen. After the user has programmed the device, the microcontroller can calculate the estimated power use of the program the user specified, then check if the connected battery is capable of providing enough power to successfully run the specified regimen. If it cannot, the user will be notified, either by the display on the programmer during programming, or by an integrated LED on the controller, which can signal the user of an error. In one aspect, the microcontroller can be programmed by the Arduino programming language where the software has been designed to specifically take advantage of power-saving features.

Referring back to FIG. 2B, in one embodiment the AAU can further comprise a pressure sensor 220. The pressure sensor 220 can be used to monitor the pressure between the pneumatic pump 204 and the nebulizer jet 206 to enable feedback that can aid in maintaining an accurate dispersal during a battery-drain event. A drop in pressure during operation is indicative of the battery losing power. Without correction, this drop in power, and resulting drop in pressure, will produce less aerosol, which will yield a lower gaseous concentration than desired. If a drop in pressure is detected, the duration of nebulizing can be extended by an appropriate amount of time to ensure the dispersal will yield the desired gaseous concentration.

In one aspect, a web-application can be used that takes user input for a treatment regimen and creates a correctly-formatted configuration file to be placed on the SD card 212, to be read by the device. This ensures that the configuration file contains no formatting errors that would cause aberrant behavior of the AAU. If there is an issue with user input, the web-application notifies the user before the configuration file is generated, and allows the user a chance to correct the issue before the configuration file is generated.

Volatile Organic Compounds

In some embodiments, the gaseous compounds delivered by the disclosed AAU are antifungal compounds. In some cases, the gaseous compounds are volatile organic compounds (VOC) or VOC formulations. VOCs are organic chemicals that have a high vapor pressure at ordinary room temperature. Their high vapor pressure results from a low boiling point, which causes large numbers of molecules to evaporate or sublimate from the liquid of the compound and enter the surrounding air.

For example, the VOC can be a fatty alcohol having between 4 and 12 carbon atoms such as 2-ethyl-1-hexanol, 3-nonanol, 1-octen-3-ol, hexanol, 3-methyl-1-butanol, isobutanol, 3-octanol, (Z)-3-hexen-1-ol, 1-penten-3-ol, ethanol, isomers, derivatives and mixtures thereof, as well as cyclic alcohols such as menthol or compounds derived from phenols such as phenylethanol, phenylmethanol, 2,4-di-t-butylphenol, isomers, derivatives and mixtures thereof or compounds derived from terpene such as isoborneol, 2-methyl isoborneol, 2-norbonanol, cariophyllene, aristolene, α-bergamotene, naphthalene, α-patchoulene, myrcene, a- and b-phellandrene, limonene, linalool, carvacrol, thymol, camphene, geraniol, nerol, and derivatives and mixtures thereof.

In particular embodiments, the VOCs can be selected from the group consisting of 2-ethyl-1-hexanol, benzaldehyde, benzothiazole, decanal, nonanal, N,N-dimethyloctylamine, propionoic acid, 2-nonanone, undecene, styrene, β-phenylethanol, and dimethyl sulfide. The VOCs can be combined to increase effectiveness. For example, the liquid to be dispersed in aerosol form can comprise 2-ethyl-1-hexanol and benzaldehyde; 2-ethyl-1-hexanol and nonanal; 2-ethyl-1-hexanol and decanal; or 2-ethyl-1-hexanol and N,N-dimethyloctylamine. In some cases, the liquid to be dispersed in aerosol form comprises 2-ethyl-1-hexanol, benzaldehyde, and decanal. In some cases, the liquid to be dispersed in aerosol form comprises 2-ethyl-1-hexanol, nonanal, and decanal.

In some embodiments, the VOCs comprise antimicrobial compounds from a Muscodor species as described in U.S. Pat. No. 8,728,462, which is hereby incorporated by reference in its entirety for the teaching of these compounds. A synthetic formulation of the VOCs produced by the fungus Muscodor crispans strain B23, further referred to as B23, has demonstrated significant antimicrobial activity against a broad range of human and plant pathogens, including both fungi and bacteria (Mitchell et al., 2010 Microbiology 156:270-277). The B23 formulation has also demonstrated significant in vitro anti-P. destructans activity and, in a small field trial, did not elicit any signs of irritation or avoidance when sachets of B23-soaked vermiculite were hung in close proximity to torpid bats. The VOCs of the B23 formulation (Table 1) are currently on the US Food and Drug Administration's list of substances that are generally recognized as safe (GRAS), indicating their low toxicity. Therefore, in some cases, the VOCs can be selected from the VOCs listed in Table 1.

TABLE 1
VOCs produced by M. crispans strain B23, identified by gas chromatography mass
spectroscopy (GC/MS), that comprise the synthetic formulation (Mitchell et al., 2010).

| Retention | | | | | | |
|---|---|---|---|---|---|---|
| Time | Total | | | Ratio | Ratio | |
| (Minutes) | Area | Compound | MW | 1x | 1000x | % |
| 2:05 | 1.39 | Acetaldehyde | 44.03 | 3.96E−03 | 3.96 | 0.34% |
| 3:51 | 2.83 | 2-Butanone | 72.06 | 8.06E−03 | 8.06 | 0.69% |
| 4:08 | 30.56 | Propanoic acid, 2-methyl-, | 102.07 | 8.70E−02 | 87.02 | 7.47% |
| | | methyl ester | | | | |
| 5:29 | 2.29 | Acetic acid, 2- | 116.08 | 6.52E−03 | 6.52 | 0.56% |
| | | methylpropyl ester | | | | |
| 6:39 | 1.09 | Propanoic acid, 2-methyl-, | 144.12 | 3.10E−03 | 3.10 | 0.27% |
| | | 2-methylpropyl ester | | | | |
| 6:46 | 1.78 | 1-Propanol, 2-methyl- | 74.07 | 5.07E−03 | 5.07 | 0.44% |
| 6:52 | 1.51 | 2-Butenal, 2-methyl-,(E)- | 84.06 | 4.30E−03 | 4.30 | 0.37% |
| 7:12 | 4.79 | 1-Butanol, 3-methyl-, | 130.1 | 1.36E−02 | 13.64 | 1.17% |
| | | acetate | | | | |
| 8:21 | 4.78 | Propanoic acid, 2-methyl-, | 158.13 | 1.36E−02 | 13.61 | 1.17% |
| | | 2-methylbutyl ester | | | | |
| 8:31 | 5.38 | 1-Butanol, 3-methyl- | 88.09 | 1.53E−02 | 15.32 | 1.32% |
| 13:37 | 351.18 | Propanoic acid, 2-methyl- | 88.05 | 1.00E+00 | 1000.00 | 85.89% |
| 16:44 | 1.31 | Acetic acid, 2-phenylethyl | 164.08 | 3.73E−03 | 3.73 | 0.32% |
| | | ester | | | | |

Essential Oils

In some embodiments, the antifungal compound comprises the volatile vapor of an essential oil. The term, “essential oil” refers to a highly odoriferous, volatile liquid component obtained from plant tissue. Essential oils typically include a mixture of one or more terpenes, esters, aldehydes, ketones, alcohols, phenols, and/or oxides. These functional classes of compounds are responsible for the therapeutic properties and distinct fragrance of the essential oil.

The essential oil can be manufactured (i.e., synthesized or partially synthesized). Alternatively, the essential oil can be obtained from a plant or plant component (e.g., plant tissue). Suitable plant or plant components include, e.g., a herb, flower, fruit, seed, bark, stem, root, needle, bulb, berry, rhizome, rootstock, leaf, or a combination thereof.

The specific essential oil will preferably be non-toxic to mammals (e.g., bats) and will be suitable for veterinary use (e.g., topically). The specific essential oil will also preferably comply with any controlling or governing body of law, e.g., FDA regulations.

In some cases, the gaseous compounds are non-volatile compounds or any other liquids that can be aerosolized.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Inhibition of Pseudogymnoascus destructans Growth from Conidia and Mycelial Extension by Bacterially Produced Volatile Organic Compounds

Materials and Methods

Culture Acquisition and Maintenance

P. destructans cultures were maintained on Sabauroud Dextrose Agar (SDA) or in Sabauroud Dextrose Broth (SDB) (BD, Md.) at 4-15° C. P. destructans spores were stored in phosphate-buffered saline (PBS) at −20° C. Spores were stored no longer than 3 weeks prior to use.

VOC Exposure Assays and Evaluation of Bacterially Produced VOCs for Anti-P. destructans Activity

Volatile organic compounds previously shown to be produced by bacteria [Chuankun X, et al. Soil Biol Biochem. 2004 36:1997-2004; Fernando W G D, et al. Soil Biol Biochem. 2005 37:955-64] were screened for anti-P. destructans activity via VOC exposure to spores and mycelial plugs. The VOCs included: decanal; 2-ethyl-1-hexanol; nonanal; benzothiazole; benzaldehyde; and N,N-dimethyloctylamine (Sigma-Aldrich, Missouri). All VOCs were chosen based on their identification in fungistatic soils and their observed production in bacteria [Chuankun X, et al. Soil Biol Biochem. 2004 36:1997-2004; Fernando W G D, et al. Soil Biol Biochem. 2005 37:955-64]. All VOCs purchased as pure, liquid, research grade reagents and used directly, without modification, in all subsequent assays. A single-compartment Petri plate (150 mm×15 mm) was used for a contained airspace to assess P. destructans growth characteristics in the presence of fungistatic VOCs. Ten microliters of P. destructans conidia suspension (10⁶ conidia ml⁻¹ in PBS) was spread onto SDA plates (35 mm×10 mm). Aliquots of 30, 3.0, or 0.3 μl of each VOC corresponding to maximum possible relative concentrations ranging from 113 ppm (v/v) to 1.13 ppm (v/v) were pipetted onto a sterile filter paper disk (12.7 mm) on a watch glass (75 mm). Each VOC containing disk and watch glass was placed inside a large Petri plate (150 mm 9 15 mm) along with a P. destructans-inoculated SDA plate (35 mm×10 mm) (FIG. 6). P. destructans mycelial plugs cut from the leading edge of actively growing colonies were inserted into fresh SDA plates (35 mm×10 mm) and placed in large Petri plates (150 mm×15 mm) with each formulation or pure VOC containing paper disk and sealed with parafilm M (Sigma-Aldrich, Missouri). Plates were then incubated at 15° C. for 21 days. Unexposed cultures and the addition of activated carbon to exposure assays served as negative controls for each trial. Anti-P. destructans activity was scored on a plus/minus scale for conidia-inoculated plates, and the radial growth from mycelial plugs was used to determine percent inhibition by comparing growth area of VOC exposed plugs to unexposed controls. All assays were performed in triplicate and averaged.

VOC Formulation Assay for Anti-P. destructans Activity

VOC formulations utilizing combinations of two pure VOCs were created with all fifteen possible combinations of the six VOCs by applying volumes corresponding to 2.0 μmol of each VOC to separate absorbent disks and arranging combinations of two disks of different VOCs on a single watch glass. Volumes corresponding to 4.0 μmol of each pure VOC were used as synergism controls to determine synergism. P. destructans mycelial plugs were harvested and inserted into fresh SDA plates (35 mm×10 mm) and sealed with parafilm in large Petri plates (150 mm×15 mm) with each formulation or pure VOC. Plates were then incubated at 15° C. for 21 days as described above. Each test was conducted in triplicate. Area measurements were conducted every 2 days post-inoculation with the use of digital photography and computer analysis as described below.

Area Measurement of Radial Growth with Digital Photography and Open-Source Software

Filamentous fungi grow by hyphae elongation and not by distinct cellular division. Accordingly, measuring the difference between the area growth of control agent-exposed mycelial plugs and control plugs has been a vetted method for assessing antimicrobial susceptibility [Fernando W G D, et al. Soil Biol Biochem. 2005 37:955-64; Liu W, et al. Curr Res Bacteriol. 2008 1:128-34; Strobel G A, et al. Microbiology. 2007 153:2613-20]. The use of a ruler to measure the area of mycelial growth of filamentous fungi has its own challenges. Mycelial plugs will often grow asymmetrically, either naturally or because of exposure to the compound being tested. To provide more accurate measurement of mycelial growth, a digital photography and analysis technique was developed.

The GIMP (GNU Image Manipulation Program) is open-source, freely distributed software for image editing and authoring, compatible with GNU/Linux, Microsoft Windows, Mac OS X, Sun OpenSolaris, and FreeBSD operating systems. This software allows for the direct measurement of the number of pixels in a given selected area of a photograph. GIMP version 2.8.2 for Microsoft Windows was used at the time of this writing. A Nikon D3100 digital single lens reflex camera with an 18-55 mm lens was used to capture images. A standard three-leg tripod was used for support during capture.

The camera was attached to the tripod and aimed down to a surface to provide a consistent distance from the lens to the mycelial surface being photographed; ensuring the same pixel to millimeter ratio was retained for all acquired images. Images of mycelial plugs had their corresponding image numbers catalogued for later identification. All Petri plate agar heights were similar to ensure the focal point remained consistent as well as to retain the same pixel to distance ratio. Manual focus was activated to retain the same focal point throughout all image captures, and a remote shutter release device was used to assure stable, shake-free images were acquired.

Contrast between the growth medium and mycelium was required to obtain an accurate selection for measurement as well as to be able to discern the margin of the ruler graduation marks with GIMP. Therefore, the camera's white balance, exposure, f-stop, and ISO were adjusted to retain a consistent contrast between photograph acquisitions. A photograph of a ruler was used to set the focal point for the proceeding photographs as well as serving as a calibration device for determining the length of each pixel during image analysis.

The ruler tool was used to determine the number of pixels between two demarcations of a photographed ruler placed at the level of the agar surface in the Petri plates. The resulting pixel count was used to determine the millimeter-to-pixel ratio.

A different set of tools were necessary to measure the mycelial area. The selection tools were used to outline the margin of the mycelia. The Histogram tool was used to determine the number of pixels that comprised the selected area. The area of the selection was converted from the number of pixels to mm² with the derived number of pixels per mm and Eq. 1.

$${( \frac{Number of pixels in area}{Number of pixels per mm} )}^{2} = Area of mycelia in {mm}^{2} \qquad Eq . 1$$

Tape Mount Preparation and Microscopic Evaluation Pseudogymnoascus destructans cultures with aberrant phenotypes as compared to control cultures and published descriptions [Garbeva P, et al. Soil Biol Biochem. 2001 43:469-77] were examined microscopically by tape mount. The adhesive side of standard transparent packaging tape was gently pressed against the surface of plate grown fungal colonies. The resulting tape-adhered sample was treated with 10 μl of 70% ethanol and placed onto a microscope slide with lactophenol cotton blue dye. Slides were viewed on a light microscope (Nikon optiphot-2) at 200×magnification and images captured using a scope mounted camera (Qlmaging micropublisher 3.3 RTV).

Results

Anti-P. destructans Activity of Bacterially Produced Volatiles

Initial investigation demonstrated inhibitory activity for most VOCs at relative concentrations less than 1 ppm. Decanal; 2-ethyl-1-hexanol; nonanal; benzothaizole; dimethyltrisulfide; benzaldehyde; and N,Ndimethyloctylamine all demonstrated anti-P. destructans activity when 30 μl of the respective compound were placed adjacent to SDA plates inoculated with P. destructans conidia in a closed system at 15° C. (Table 2). Control plates containing 1 g activated carbon showed no inhibition for decanal; 2-ethyl-1-hexanol; and benzaldehyde, while the remaining compounds inhibitory activity persisted in the presence of activated carbon (Table 2). Subsequent assays with 3 μl of each compound demonstrated similar results with only N,N-dimethyloctylamine unable to completely inhibit P. destructans growth from conidia at 7 days (Table 2). The addition of activated carbon abolished all inhibitory activity of the assayed compounds at 3 μl (Table 2). At 11 days of exposure to 3 μl of each respective compound, only 2-ethyl-1-hexanol, decanal, and nonanal demonstrated inhibitory activity, with all activated carbon controls abolishing the inhibitory activity (Table 2). Additionally, P. destructans cultures from conidia exposed to 3 μl benzothiazole without activated carbon revealed unique colony morphology characterized by increased pigmentation of the underside of the culture and diffusion of pigment into the growth media as compared to unexposed cultures and cultures exposed to benzothiazole in the presence of activated carbon.

TABLE 2
Evaluation of anti-P. destructans activity of bacterially produced
antifungal VOCs with P. destrunctans condidia

| VOC | 30 μl | 30 μl^{c} | 3 μl^{a} | 3 μl^{a,c} | 3 μl^{b} | 3 μl^{b,c} |
|---|---|---|---|---|---|---|
| 2-ethyl-1-hexanol | − | + | − | + | − | + |
| Benzaldehyde | − | + | − | + | + | + |
| Benzothiazole | − | − | − | + | + | + |
| Decanal | − | + | − | + | − | + |
| Nonanal | − | − | − | + | − | + |
| N,N-dimethyloctylamine | − | − | + | + | + | + |
| Control | + | + | + | + | + | + |

+, growth from spores;
−, no visible growth
^{a}7 day exposure
^{b}10 day exposure
^{c}Incubated with activated carbon

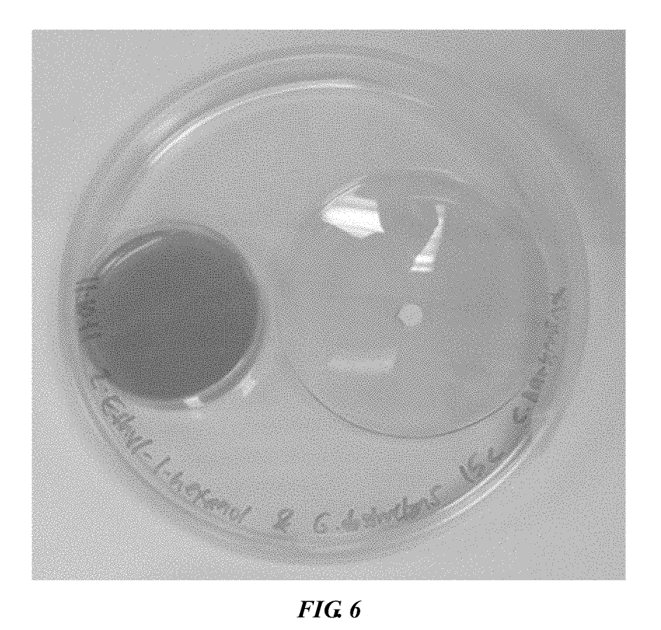

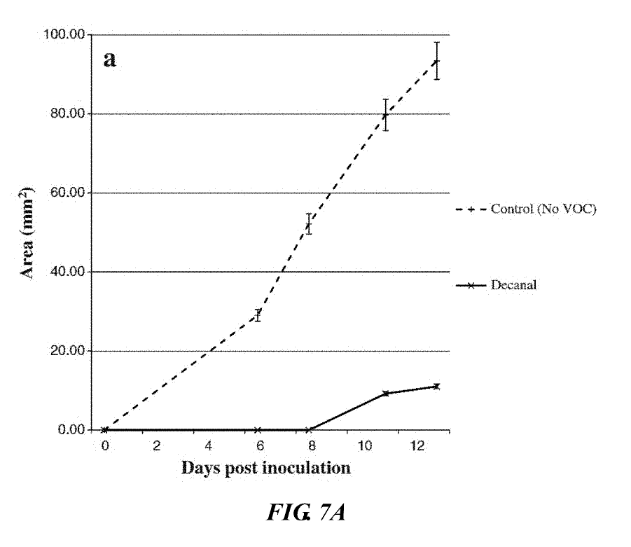

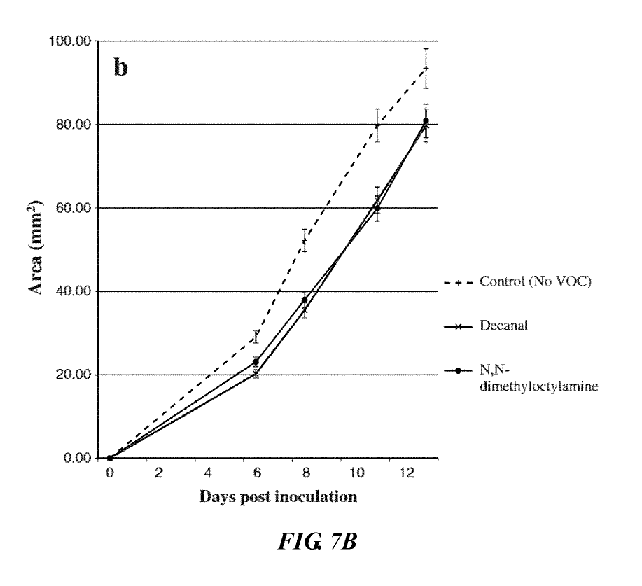

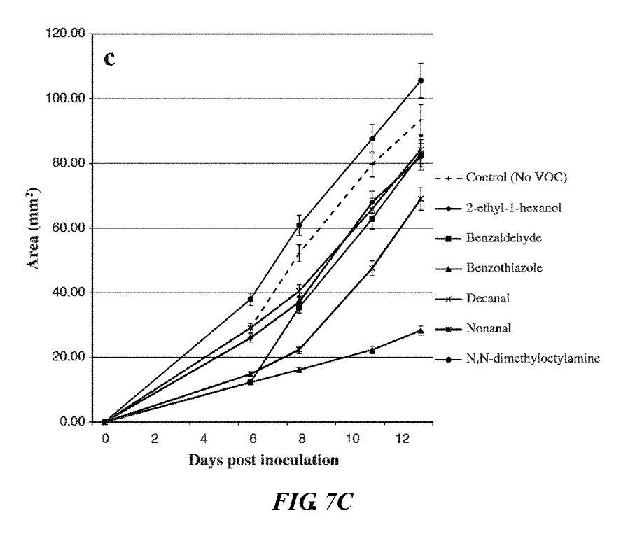

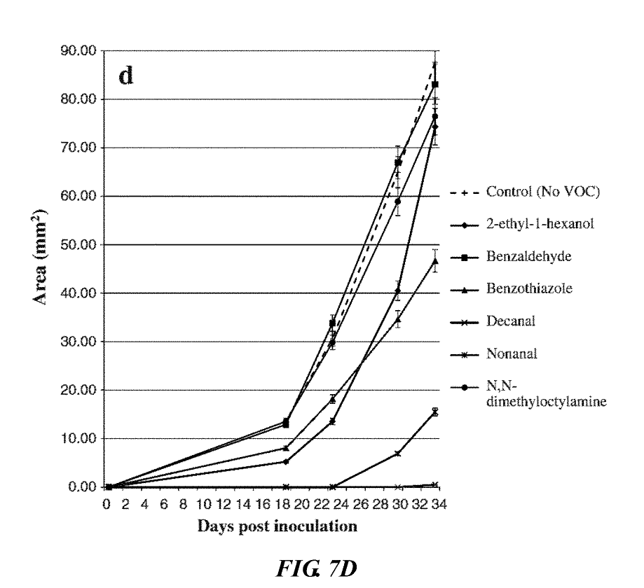

Assays using mycelial plugs cut from the leading edge of actively growing P. destructans colonies on SDA exposed to the previously described bacterially produced volatiles at 30, 3, and 0.3 μl of each respective compound and incubated in a contained air space at 15° C. gave varied results. At 30 μl, all compounds completely inhibited the growth of P. destructans mycelia for up to 9 days (FIG. 7A). At 14 days of exposure, only P. destructans plugs exposed to decanal showed any radial growth, with 83% reduction in growth as compared to unexposed controls (FIG. 7A). At 3 μl of each compound, decanal and N,N-dimethyloctylamine yielded only minor reductions in radial growth, whereas the remaining compounds completely inhibited radial mycelial growth of P. destructans for up to 14 days (FIG. 7B). At 0.3 μl of each compound, only benzothiazole demonstrated significant inhibitory activity with a 60% reduction in radial growth after 14 days of exposure (FIG. 7C). Interestingly, at 0.3 μl, N,N-dimethyloctylamine induced growth as compared to unexposed controls (FIG. 7C). This result may be due to hormesis [Stebbing A R D. Sci Total Environ. 1982 22:213-34].

In order to forecast the in situ efficacy of the VOCs additional in vitro evaluation was conducted at 4° C. to more accurately represent the environmental conditions of North American hibernacula. Exposure to 30 μl or 3.0 μl of each respective VOC completely inhibited radial growth of P. destructans for greater than 21 days. Exposure to 0.3 μl of each respective VOC inhibited radial growth for all VOCs except benzaldehyde (FIG. 7D). The greatest degree of inhibition was observed with decanal which demonstrated a greater than 99% reduction in growth area at 35 days post-inoculation (FIG. 7D). Based on these initial results, VOC exposure was standardized to 4.0 μl per headspace for subsequent evaluations. In addition to evaluating individual VOCs, formulations were investigated for potential synergistic effects.

VOC Formulations Demonstrate Synergistic Anti-P. destructans Activity

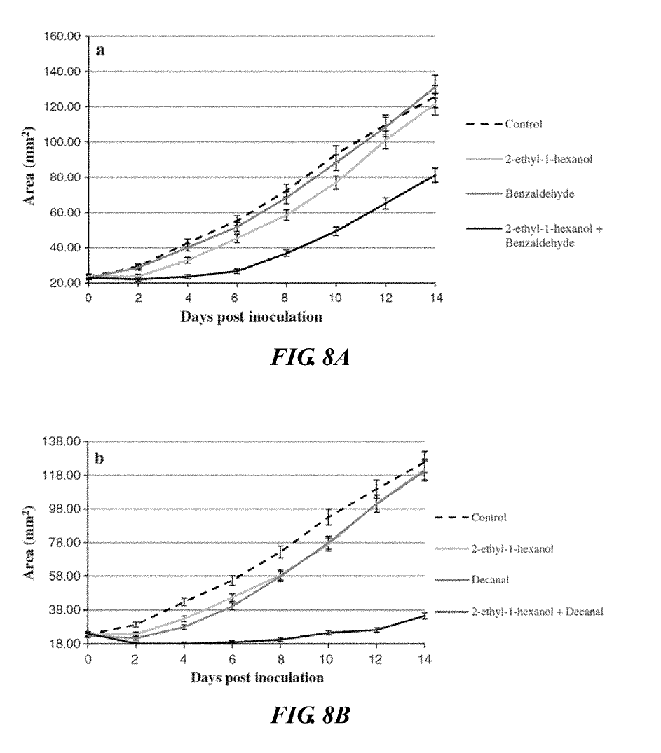

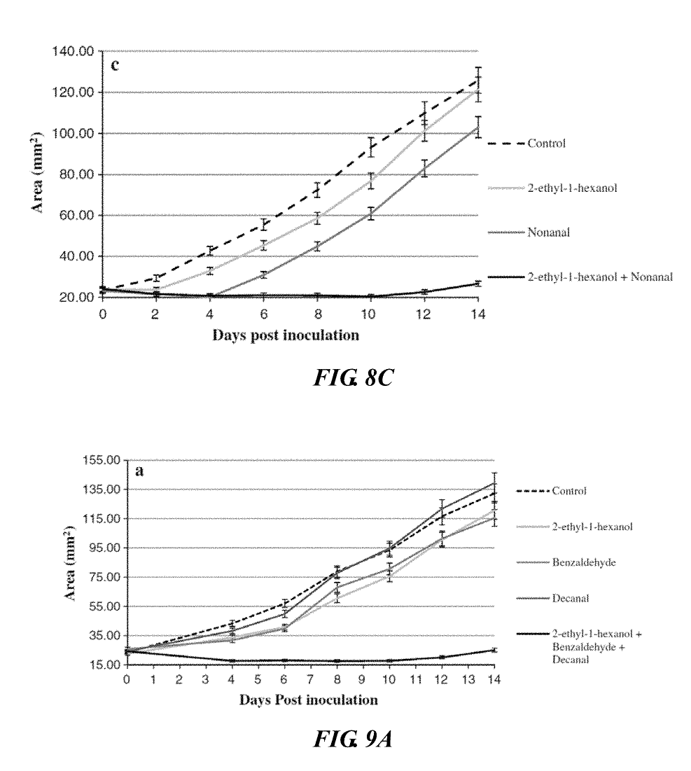

Three VOC formulations comprised of two VOCs were observed to synergistically inhibit the growth of P. destructans mycelial plugs, more than the combined inhibition of each of the pure VOCs alone. Those include 2-ethyl-1-hexanol and benzaldehyde; 2-ethyl-1-hexanol and nonanal; 2-ethyl-1-hexanol and decanal; and 2-ethyl-1-hexanol and N,N-dimethyloctylamine (FIG. 8A, 8B, 8C, respectively). The greatest inhibition by the formulation occurred with 2-ethyl-1-hexanol and nonanal, which demonstrated greater than 95% reduction in growth as compared to unexposed controls 14 days post-inoculation (FIG. 8C).

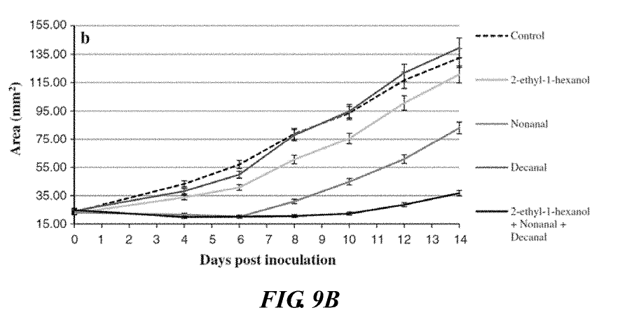

Two VOC formulations comprised of three VOCs at 1.33 μma respectively, were observed to synergistically inhibit the growth of P. destructans mycelial plugs, more than the combined inhibition of each of the pure VOCs alone at 4.0 μma Those include 2-ethyl-1-hexanol; benzaldehyde; and decanal; as well as 2-ethyl-1-hexanol; nonanal; and decanal (FIG. 9A, 9B).

### Example 2: Development of an Automated VOC Dispersal Device to Distribute Antifungal VOCs and their Formulations for the Treatment and Prevention of White-Nose Syndrome

Materials and Methods

VOC Dispersal Device Development, an Automated Aerosolization Unit (AAU).

Real circuits were developed on prototyping breadboards to assess viability of theorized circuit and programming designs. An ATMEGA168-PA-PU AVR microcontroller unit (MCU) (Atmel, Calif.) was selected as the processor and an nMOSFET, part FQP30N06L (Fairchild Semiconductor, California), was selected to control the nebulizer activity. Breadboard circuit prototypes were tested under various conditions likely to be experienced under normal operation, before printed circuit boards (PCBs) prototypes were developed (Fritzing, United Kingdom).

Software Programming of AAU

The ATMEGA microprocessor was programmed with the Arduino programming language and compiled with the Arduino Integrated Development Environment (IDE), version 1.0.5 to produce a hex file containing the machine code. The hex code was uploaded to the ATMEGA either with the Arduino IDE or AVRDude (FOSS, Brian S. Dean) software, using the Pocket AVR Programmer (Sparkfun Electronics, Colorado).

Ethanol aerosolization by AAU.

A Trek S Portable Aerosol System (PARI Respiratory Equipment, Virginia) was connected to AAU. A container with an inner volume of 0.2183 m³ was used for concentration testing. 3 ml of ethanol was loaded into the pump reservoir. The reservoir was weighted before and after aerosolization into the container. A HAS-301-1050A quantitative gas analysis (QGA) mass spectrometer (Hiden Analytical, United Kingdom) analyzed gas from the container to assess the change in partial pressure.

Formulation Aerosolization by AAU

After initial testing with ethanol, evaluation of the disclosed VOC formulation began to determine if the aerosolization characteristics differed between compounds. 3 ml of formulation was loaded into the reservoir of the nebulizer. AAU powered the nebulizer for various durations of time, ranging from 3 to 14 seconds. Reservoir weight measurements were taken before and after each application.

Timing Accuracy of AAU

A real time clock (RTC) was used to assess the disparity between the perceived duration of time (measured by AAU) and the actual duration of time (measured by the RTC). At a sustained temperature, varying voltages were applied to power the AAU as the perceived and actual durations of time were recorded. This was repeated at various temperatures and voltages likely to be encountered in the lab and field. To obtain a valid result from a multiple linear regression analysis, the data must hold true to eight assumptions: (1) the dependent variable must be measured on a continuous scale, (2) there are two or more independent variables, (3) there is independence of observation, (4) there is a linear relationship between (a) the dependent variable and each independent variable, and (b) the dependent variable and the independent variables collectively, (5) the data needs to show homoscedasticity, (6) the data must not show multicollinearity, (7) there are no significant outliers, and (8) the residuals (errors) are approximately normally distributed. SPSS Statistics (IBM, version 21) was used to assess if the data had passed all eight assumptions before producing a model.

VOC Bat Toxicity Assessment.

One species of torpid bat (n=18) were exposed to a 10 ppm gaseous VOC formulation. This was repeated every 24 hours for 42 days. A random segment of both control and test groups were removed at days 10 and 42, euthanized, and subject to a full necropsy with particular interest in the condition of respiratory tissues.

Results

VOC dispersal device development, an Automated Aerosolization Unit (AAU)

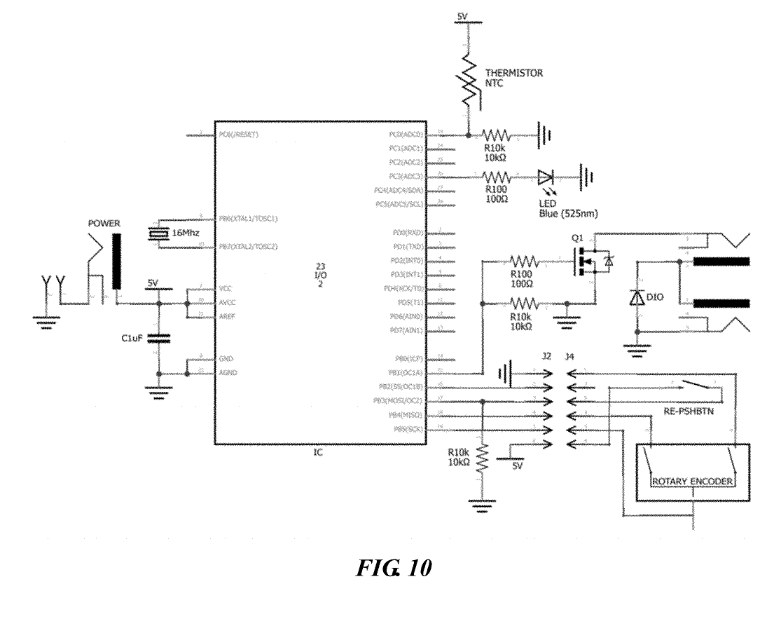

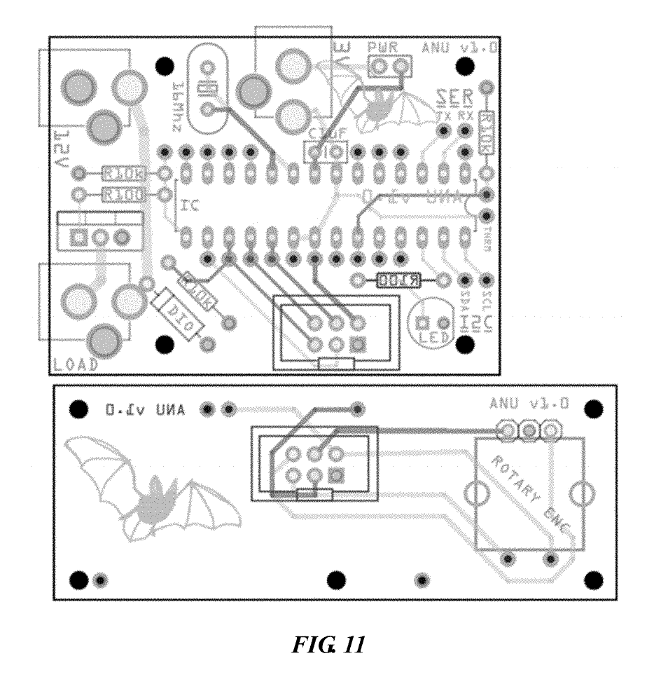

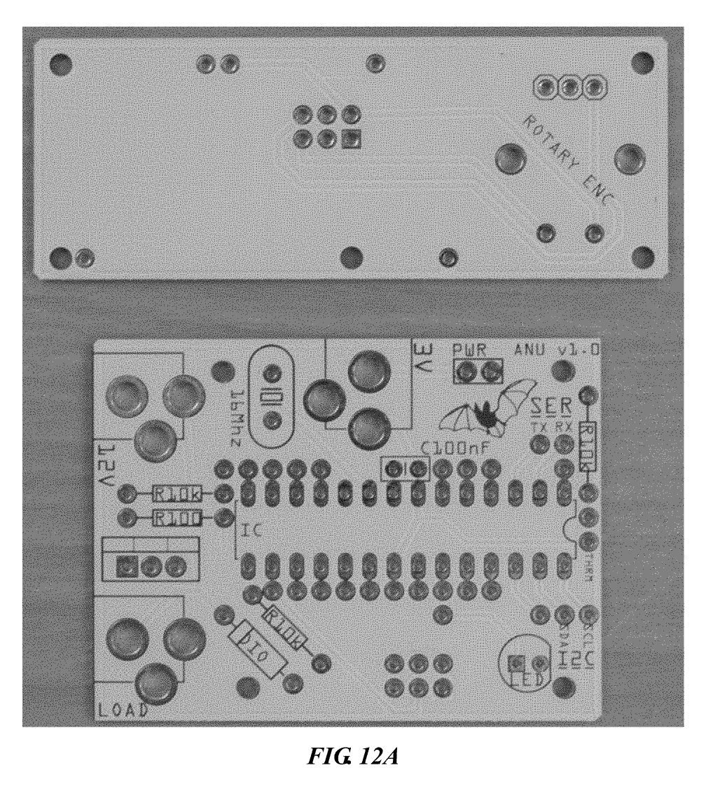

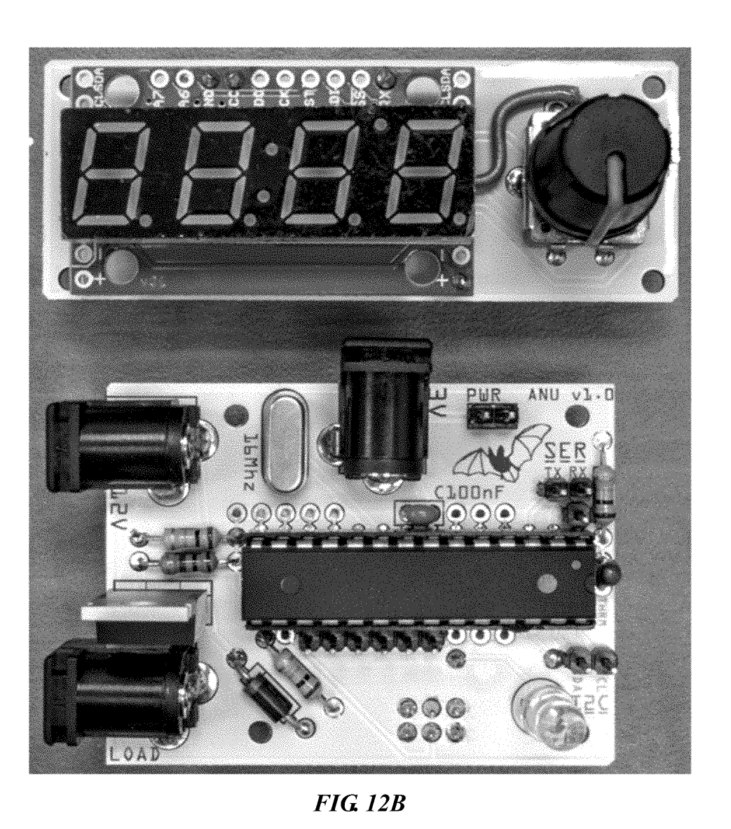

A circuit design is shown in a schematic view and a PCB view (FIGS. 10 and 11, respectively). A color photo was taken of the front face of the boards as well as with them populated with parts (FIG. 12).

Software Programming of AAU

Arduino source code to program the ATMEGA168 microcontroller is shown in Source Code 1, below, and a flow chart illustrating function interaction is shown in FIG. 4.

Ethanol Aerosolization by AAU

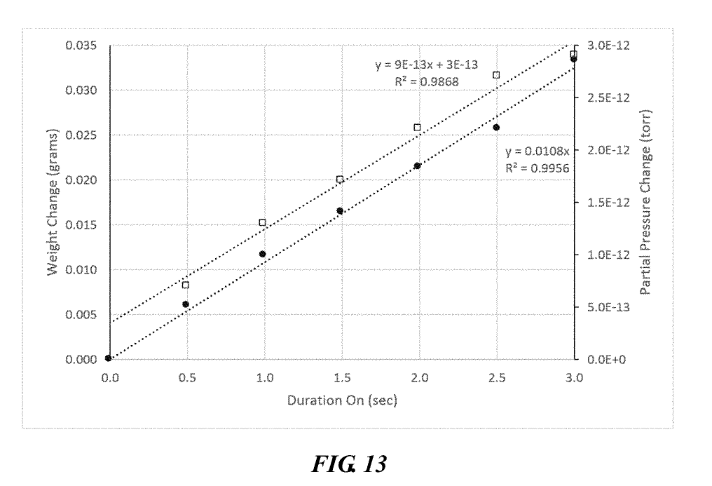

Both change in weight and change in partial pressure of ethanol are linearly related to the duration of aerosolization (FIG. 13).

Formulation Aerosolization by AAU

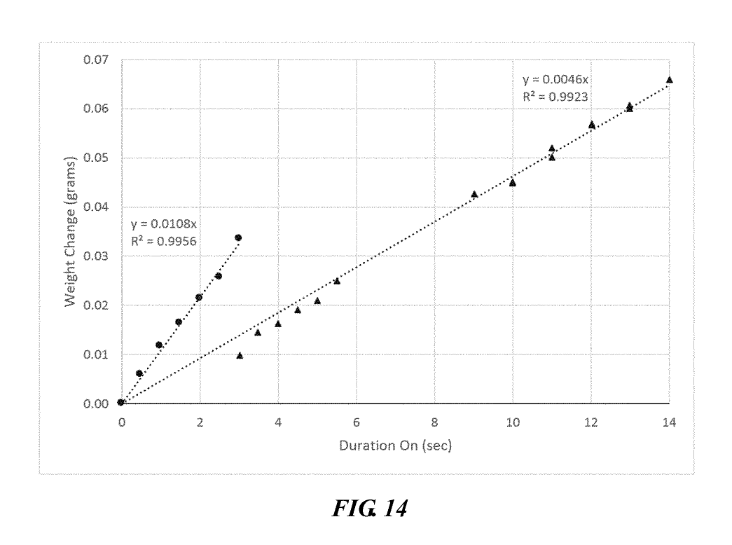

Both ethanol and the formulation demonstrate a linear relationship between change in weight and duration of aerosolization, however the slope of each differs (FIG. 14).

Timing Accuracy of AAU

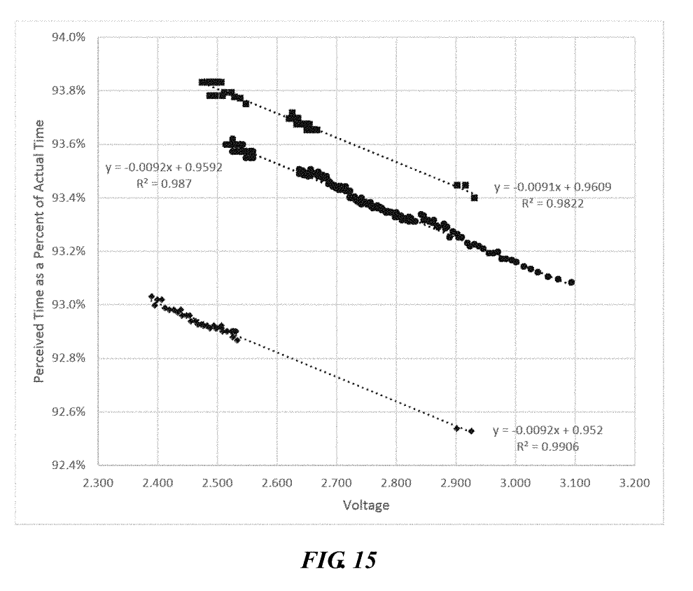

A scatter plot was produced from the collected data, comparing voltage with the perceived duration of time difference (as the % of actual duration of time) at 5° C., 15° C., and 30° C. (FIG. 15). There were no conflicts with the multiple linear regression assumptions and a model was produced from a multiple linear regression analysis, using temperature and voltage to predict a conversion factor to be applied to the perceived duration of time. Percent of actual duration of time was derived by Eq. 2, where both perceived duration of time and actual duration of time were measured in seconds. The conversion factor to apply to the perceived duration of time was derived from SPSS (Eq. 3), and produces a close approximation of the actual duration of time.

$$\begin{matrix}\% of real time = \frac{preceived time}{real time} & Eq . 2 \\ Conversion factor = ( T * 43.032292 ) + ( V * 0.939652 ) + 103999.631684 \\ T = Temperature ( ° C . ) , V = voltage & Eq . 3\end{matrix}$$

VOC Bat Toxicity Assessment

The bat toxicity trial has ended and we await results from the necroscopies to determine any ill effects from the VOC formulation.

Discussion

Classic disease management practices applied in agriculture such as broad spectrum dissemination of antibiotics are not realistic options for management of disease in wild, highly disseminated, and migratory animal populations. Accordingly, the development of an automated aerosolization unit (AAU) was undertaken that seeks to avert the spread of this disease and reduce the mortality associated with currently infected hibernacula.

The biological origin of many fungistatic VOCs lends itself to obtainable inhibitory applications due to the typically low level of production in the natural hosts and the significant antagonistic activity observed at these low levels [Chuankun X., et al. 2004. Soil Biol. Biochem. 36:1997-2004; Ezra D. and Strobel G. A. 2003. Plant Sci. 165:1229-38; Garbeva P., et al. 2001. Soil Biol Biochem. 43:469-77; Kerr J. R. 1999. Microb. Ecol. Health Dis. 11:129-42; Stebbing A. R. D. 1982. Sci Total Environ. 22:213-34]. The contact-independent activity of antagonistic VOCs has several advantages over topical and oral, contact dependent, treatment options that have been shown to be highly effective at inhibiting the growth of P. destructans in previous studies [Strobel, G. A., et al. 2001. Microbiol. 147: 2943-2950]. Contact-independent antagonisms allow for treatment of many individuals with a single application and ensures uniform exposure, avoiding the potential for microbial refugia on the host that may facilitate re-colonization of the host once the inhibitory compound has been removed or degraded.

The convolution of soil microbiota, plant associated endophytes and fungal pathogens have produced antagonisms ideally suited for the complex ecology of these environments. The long-term efficacy of low quantities of VOCs illustrates the potential of these compounds for in situ application in the treatment of WNS [Cornelison C. T., et al. 2013. Mycopathologia 177(1-2):1-10]. Additionally, the development of synergistic blends bolsters the appeal of soil-based fungistasis as a source of potential control agents as VOC mixtures are likely responsible for the observed fungistatic activity of repressive soils [Garbeva P., et al. 2001. Soil Biol Biochem. 43:469-77; Kerr J. R. 1999. Microb. Ecol. Health Dis. 11:129-42]. The evaluation of bacterially derived VOCs has expanded the pool of potential biological control agents as well produced several VOC formulations with excellent anti-P. destructans activity. The availability of volatile formulations for control of P. destructans growth could prove to be a powerful tool for wildlife management agencies if appropriate application methods can be developed.

Current technology for dissemination of VOCs and essential oils for control of odors and pests in indoor environments is common. Manufacturers' claims vary significantly depending on the product, with treatment capacities varying from 6,000 to 50,000 ft³ for a single unit, and maintaining 1-10 ppm concentrations in that area based on timed releases. Although these claims are promising, appropriate scientific validation and demonstrable consistency is lacking.

The AAU has been developed to specifically address these challenges. In addition, the design of this device allows for a wide range of essential oils, VOCs, or VOC formulations to be used. Several features have been incorporated into the design of AAU that make it ideal for mass production and use in the laboratory or field. These include (1) being built from inexpensive, readily accessible, and available parts; (2) efficient circuit design and programming that enhances low energy consumption to allow running for several months on battery power; (3) a user-friendly control interface that allows quick and easy reprogramming; (4) incorporation of a medical nebulizer that allows dispersal of highly-concentrated VOCs; (5) a modular, scalable, design that allows for use in expansive air volumes; and (6) being housed in a durable, acoustic dampening, enclosure to prevent dust and moisture intrusion as well as to limit the acoustic output that may disturb torpid bats.

Initial testing involved the aerosolization of ethanol to determine if there was a linear correlation between the duration of aerosolization and the amount of compound aerosolizes. Indeed there appeared to be a linear relationship. Furthermore, partial pressure analysis confirmed a linear relationship between duration of aerosolization and partial pressure change. A formulation also showed a linear relationship between amount aerosolizes and duration of aerosolization, however with a different slope than ethanol, suggesting that compounds or formulations with different physical properties (molecular weight, vapor pressure, etc.) will exhibit different rates of aerosolization.

Voltage and temperature were determined to be the two independent variables that contributed to the disparity between real and perceived duration of time. To adjust for this, a multiple regression analysis was conducted on the recorded voltage, temperature, perceived duration of time, and actual duration of time data. SPSS Statistics (IBM, version 21) was used to perform a multiple linear regression analysis to predict a conversion factor (dependent variable) to be applied to the perceived duration of time, from voltage and temperature (independent variables).

The ideal gas law (Eq. 4) was adapted (Eq. 5) to be able to calculate the required amount of compound to produce a specific gaseous concentration, with consideration to variables M, V, P, R, and T. To demonstrate, Eq. 6 calculates that 41.1 mg of ethanol is required to attain a 10 ppm concentration in a 2 m³ airspace at standard temperature and pressure (STP). This equation is a proof of concept that is able to be incorporated into the programming of AAU to automatically calculate the proper on/off duration of aerosolization from only entering in the required variables.

$$\begin{matrix}PV = nRT & Eq . 4 \\ \frac{( \frac{MPV}{RT} )}{1000000} = grams of compound to produce 1 ppm \\ M = molecular weight of compound ( g {mol}^{- 1} ) , \\ P = pressure ( atm ) , V = volume ( {m}^{3} ) \\ R = ideal gas constant 8.205736 * {10}^{- 5} ( {m}^{2} atm {K}^{- 1} {mol}^{- 1} ) , \\ and \\ T = temperature ( K ) & Eq . 5\end{matrix}$$

Grams of ethanol required to attain a 10 ppm gaseous concentration in 2 m³ at 1 atm and 273.15 K (0° C.):

$$[ \frac{( \frac{46.07 g {mol}^{- 1} \times 1.0 atm \times 2 {m}^{3}}{\begin{matrix}8.205736 \times \\ {10}^{- 5} {m}^{3} atm {K}^{- 1} {mol}^{- 1} \times 2.73 .15 K\end{matrix}} )}{1000000} ] \times 10 = 0.0411 grams ethanol \qquad Eq . 6$$

### Example 3

Materials and Methods

AAU Development.

Circuits were developed on prototyping breadboards to assess viability of theorized circuit and programming designs. An ATMEGA328P-PU AVR microcontroller unit (MCU) (Atmel, Calif.) was selected as the processor and an N-MOSFET was selected to control the power to the nebulizer. Breadboard circuit prototypes were tested under various conditions likely to be experienced under normal operation, before printed circuit boards (PCBs) prototypes were developed (Fritzing, United Kingdom).

Software Programming of the AAU.

The ATMEGA microprocessor was programmed with the Arduino programming language and compiled with the Arduino Integrated Development Environment (IDE), version 1.0.5 to produce a hex file containing the machine code. The hex code was uploaded to the ATMEGA either with the Arduino IDE or AVRDude (FOSS, Brian S. Dean) software, using the Pocket AVR Programmer (Sparkfun Electronics, Colorado).

VOC Bat Toxicity Assessment.

Tom Tomasi of MSU (Springfield, Mo.) exposed one species of torpid bat (n=37) to a 10 ppmv gaseous VOC formulation that previously demonstrated the highest inhibitory effects of those formulations that were tested (10-times higher than the 1 ppmv effective dose) [Cornelison C. T., et al. 2013. Mycopathologia 177(1-2):1-10]. This was repeated every 24 hours for 42 days. A random segment of both control and test groups were removed at days 10 (acute exposure) and 42 (chronic exposure), euthanized, and subject to a full necropsy by M. Kevin Keel at UC Davis (Davis, Calif.), with particular interest in the condition of respiratory tissues. The tissues were analyzed for autolysis/putrefaction, erosive esophagitis, neutrophilic conjunctivitis, cornea neutrophilic keratitis, multifocal steatitis/necrosis of fat, atrophy of fat, acute colitis (fungi) of the large intestine, acute (neutrophilic) colitis of the large intestine, granulomatous enteritis (nematode) of the small intestine, neutrophilic enteritis (bacteria) of the large intestine, coccidosis of the small intestine, trematodiasis of the small intestine, intralumenal hemorrhage of the ntestines/stomach, focal granulomatous interstitial nephritis of the kidney, granulomatous serositis of the kidney, Circulating neutrophilia of the lung, neutrophilic bronchitis/tracheitis of the lung, hemorrhage/edema of the lung, congestion of the lung, histiocytic pulmonary hemosiderosis of the lung, neutrophilic interstitial pneumonia of the lung, inflammatory drainage reaction of the mediastinal lymph node, neutrohilic pancreatitis with chronic steatitis & serositis of the ancreas, acariasis (pres Demodex spp.) of the muzzle skin, dermatophytosis of the muzzle skin, neutrophilic dermatitis of the muzzle skin, and elevation of keratinized epithelium of the patagium skin.

Results

AAU Development

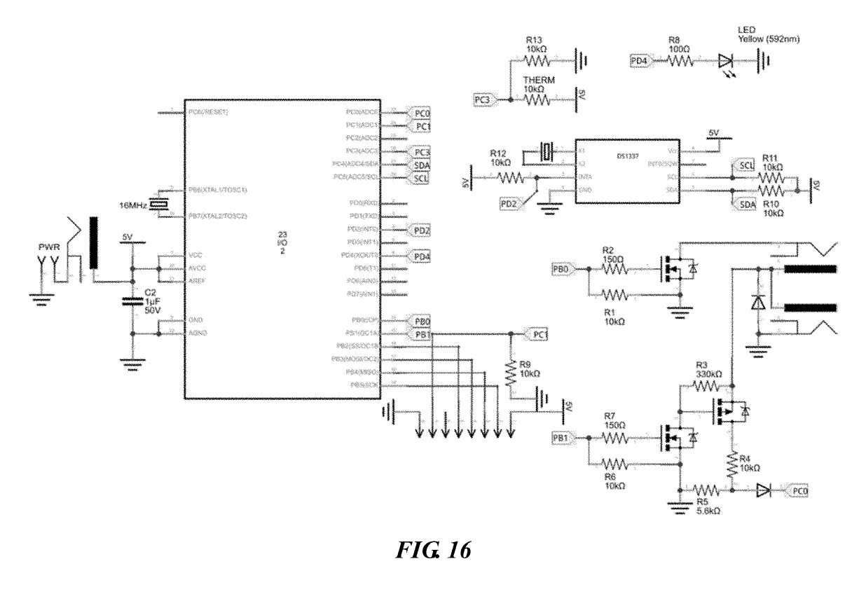

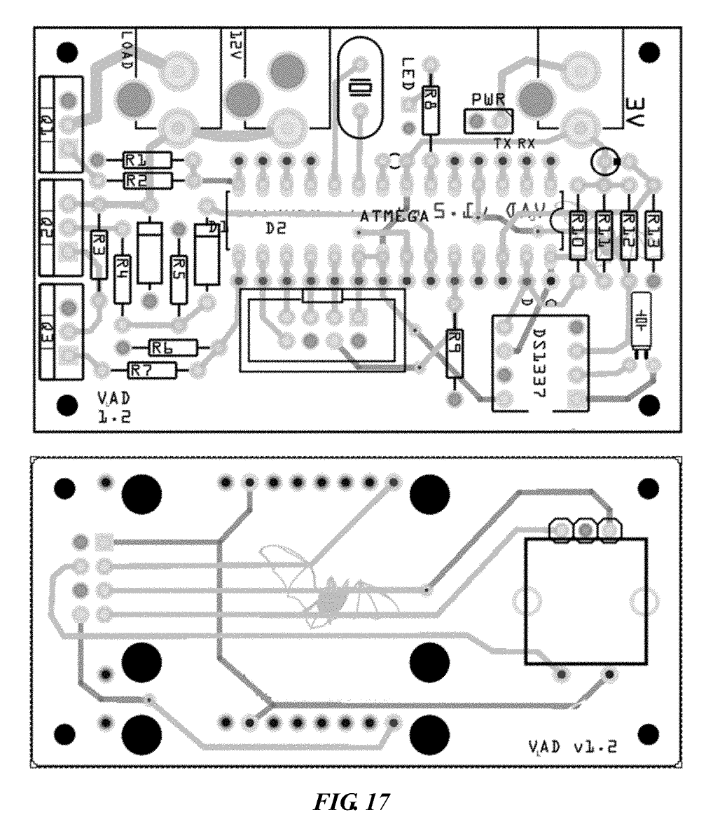

A circuit board revision of the controller is rendered in a schematic view (FIG. 16) and the controller and programmer in a PCB view (FIG. 17). Changes from prior versions include replacing the ATMEGA168 with an ATMEGA328, rearrangement of mounting holes for better fitment in 3D-printed cases, the addition of a DS1337 module for accurate time keeping, a new circuit to monitor the voltage of the nebulizer battery, changing the DC barrel plug sizes to prevent incorrectly connecting a plug that would damage the low-voltage microcontroller, changing the LED resistor value to lower its current draw, the addition of a second display on the programmer, replacing the 6-pin connector with an 8-pin connector to allow communication to the second display on the programmer, replacing the straight headers with right-angle headers to reduce the case height, part rearrangements, and other minor fixes and improvements.

Software Programming of the AAU

Arduino source code to program the ATMEGA328 microcontroller can be found in Source Code 2. A flow chart has been created to illustrate program function (FIG. 18).

VOC Bat Toxicity Assessment

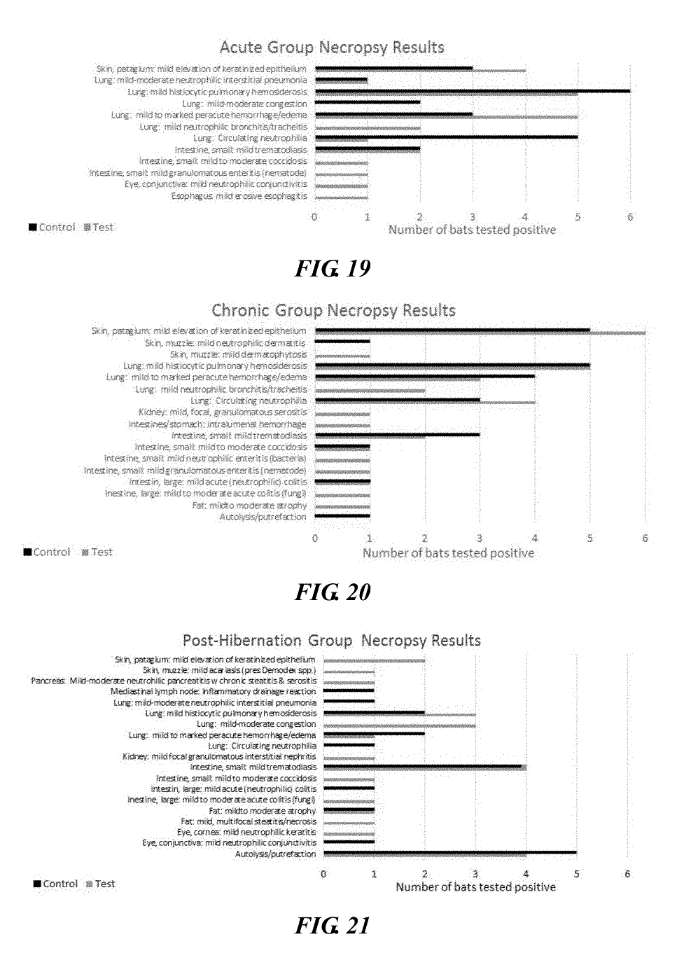

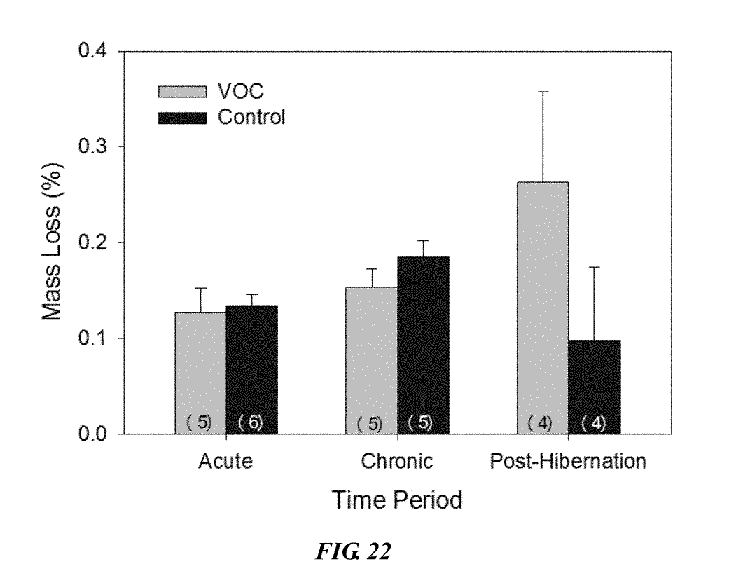

No statistically significant toxicological effects were observed among the acutely- and chronically-exposed bats (FIGS. 19 and 20). Circulating neutrophilia of lung tissue was elevated in the control group when compared to the acutely-exposed group (p=0.009). Post-hibernation weight loss was greater within the test group, however only a graph is available until the raw data has been received (FIG. 21).

Discussion

Classic disease management practices applied in agriculture such as broad spectrum dissemination of antibiotics are not realistic options for management of disease in wild, highly disseminated, and migratory animal populations. Accordingly, the development of a novel treatment option, an automated aerosolization unit (AAU), was undertaken that seeks to avert the spread of this disease and reduce the mortality associated with currently infected hibernacula. To this end, evaluation of previously described bacterially produced antifungal volatile formulation was conducted with this newly-developed VOC dispersal device, in vitro and an in vivo toxicity assay.

The biological origin of many fungistatic VOCs lends itself to obtainable inhibitory applications due to the typically low level of production in the natural hosts and the significant antagonistic activity observed at these low levels [Chuankun X., et al. 2004. Soil Biol. Biochem. 36:1997-2004; Ezra D. and Strobel G. A. 2003. Plant Sci. 165:1229-38; Garbeva P., et al. 2001. Soil Biol Biochem. 43:469-77; Kerr J. R. 1999. Microb. Ecol. Health Dis. 11:129-42; Stebbing A. R. D. 1982. Sci Total Environ. 22:213-34]. The contact-independent activity of antagonistic VOCs has several advantages over topical and oral, contact dependent, treatment options that have been shown to be highly effective at inhibiting the growth of P. destructans in previous studies [Strobel, G. A et al. 2001. Microbiol. 147: 2943-2950]. Contact-independent antagonisms allow for treatment of many individuals with a single application and ensures uniform exposure, avoiding the potential for microbial refugia on the host that may facilitate re-colonization of the host once the inhibitory compound has been removed or degraded.

The coevolution of soil microbiota, plant associated endophytes and fungal pathogens have produced antagonisms ideally suited for the complex ecology of these environments. The long-term efficacy of low quantities of VOCs illustrates the potential of these compounds for in situ application in the treatment of WNS [Cornelison C. T., et al. 2013. Mycopathologia 177(1-2):1-10]. Additionally, the development of synergistic blends bolsters the appeal of soil-based fungistasis as a source of potential control agents as VOC mixtures are likely responsible for the observed fungistatic activity of repressive soils [Garbeva P., et al. 2001. Soil Biol Biochem. 43:469-77; Kerr J. R. 1999. Microb. Ecol. Health Dis. 11:129-42]. The evaluation of bacterially derived VOCs has expanded the pool of potential biological control agents as well produced several VOC formulations with excellent anti-P. destructans activity [Cornelison C. T., et al. 2013. Mycopathologia 177(1-2):1-10]. The availability of volatile formulations for control of P. destructans growth could prove to be a powerful tool for wildlife management agencies if appropriate application methods can be developed.

Current technology for dissemination of VOCs and essential oils for control of odors and pests in indoor environments is common. Manufacturers' claims vary significantly depending on the product, with treatment capacities varying from 6,000 to 50,000 ft³ for a single unit, and maintaining 1-10 ppm concentrations in that area based on timed releases. Although these claims are promising, appropriate scientific validation and demonstrable consistency is lacking.

The AAD has been developed to specifically address these challenges. In addition, the design of this device allows for a wide range of essential oils, VOCs, or VOC formulations to be used. Several features have been incorporated into the design of the AAD that make it ideal for mass production and use in the laboratory or field. These include (1) being built from inexpensive, readily accessible, and available parts; (2) efficient circuit design and programming that enhances low energy consumption to allow running for several months on battery power; (3) a user-friendly control interface that allows quick and easy reprogramming; (4) incorporation of a medical nebulizer that allows dispersal of highly-concentrated VOCs; (5) a modular, scalable, design that allows for use in expansive air volumes; and (6) being housed in a durable, acoustic dampening, enclosure to prevent dust and moisture intrusion as well as to limit the acoustic output that may disturb torpid bats.

The ideal gas law (Eq. 4) was adapted (Eq. 5) to be able to calculate the required amount of compound to produce a specific gaseous concentration, with consideration to variables M, V, P, R, and T. To demonstrate, equation 6 calculates that 41.1 mg of ethanol is required to attain a 10 ppm concentration in a 2 m³ airspace at standard temperature and pressure (STP). This equation is a proof of concept that is able to be incorporated into the programming of the VADD to automatically calculate the proper on/off interval of aerosolization by entering in the required variables.

$$\begin{matrix}PV = nRT & Equation 4 \\ \frac{MPV}{RT} \times \frac{1}{1 , 000 , 000} = grams of compound to produce 1 ppm \\ M = molecular weight of compound ( g {mol}^{- 1} ) \\ P = pressure ( atm ) \\ V = volume ( {m}^{3} ) \\ R = ideal gas constant 8.205736 * {10}^{- 5} ( {m}^{2} atm {K}^{- 1} {mol}^{- 1} ) \\ and \\ T = temperature ( K ) & Equation 5\end{matrix}$$

Grams of ethanol required to attain a 10 ppmv gaseous concentration in 2 m³ at 1 atm and 273.15 K (0° C.):

$$\begin{matrix}\frac{46.07 g {mol}^{- 1} \times 1.0 atm \times 2 {m}^{3}}{\begin{matrix}8.205736 \times \\ {10}^{- 5} {m}^{3} atm {K}^{- 1} {mol}^{- 1} \times 2.73 .15 K\end{matrix}} \times \frac{10}{1 , 000 , 000} = 0.0411 grams ethanol & Equation 6 \\ [ Seconds of nebulization ] = [ weight of ethanol ] / 0.0079 & Equation 7\end{matrix}$$

The slope of the linear relationship between nebulization time and amount of ethanol nebulized was empirically-derived (Eq. 7). From equation 3, if 0.0411 grams of ethanol is required for the desired gaseous concentration, equation 4 states that the nebulizer would be required to run for 5.2025 seconds. If the air turnover rate is 1 m³ per hour, this is the duration the nebulizer will need to run every 2 hours to return the concentration to 10 ppmv. However, nearing 2 hours post-nebulization, the concentration will be reaching 0 ppmv. A minimum concentration can be established by nebulizing before 100% of the air has been evacuated from the air space. After the initial nebulization duration to attain the desired 10 ppmv, a reduced off duration can be applied to maintain a specific lower ppmv (Table 3). By reducing the off time by ½, to sustain a minimum ppmv of 5 ppm, the on time must also be reduced by ½, the ensure the upper ppmv will be 10 ppmv.

TABLE 3
Nebulizer off and on durations to maintain a certain lower
ppmv, after an initial on duration to raise the 2 m³ airspace to 10
ppmv, given a 100% air turnover rate is 1 m³/hour.

| Minutes Off | Seconds On | Lower ppmv | Max ppmv |
|---|---|---|---|
| 120 | 5.2025 | 0 | 10 |
| 60 | 2.6013 | 5 | 10 |
| 30 | 1.3006 | 7.5 | 10 |
| 15 | 0.6503 | 8.75 | 10 |

Within air spaces that maintain a specific airflow, this will consistently maintain a 5-10 ppmv concentration. Every hour the concentration will drop from 10 ppmv to 5 ppmv; at which time the nebulizer will bring the concentration back to 10 ppmv. However, if the airflow varies, this can cause an increase of decrease in ppmv. To improve accuracy, it may be beneficial to either decrease the off duration, allowing the on duration to raise a larger ppmv, or to incorporate a sensor to measure airflow near the nebulizer and adjust for airflow fluctuations.

An early prototype with the most effective VOC formulation from previous research [Cornelison C. T., et al. 2013. Mycopathologia 177(1-2):1-10] has recently completed toxicity trials on torpid bats at Missouri State University. The results reveal no significant toxicity when compared to the controls, even at 10-times the effective concentration. Although not all data has been recovered and analyzed, preliminary findings suggest this formulation and application method to be a promising tool for future infectivity studies.

### Example 4

Results

AAU Development

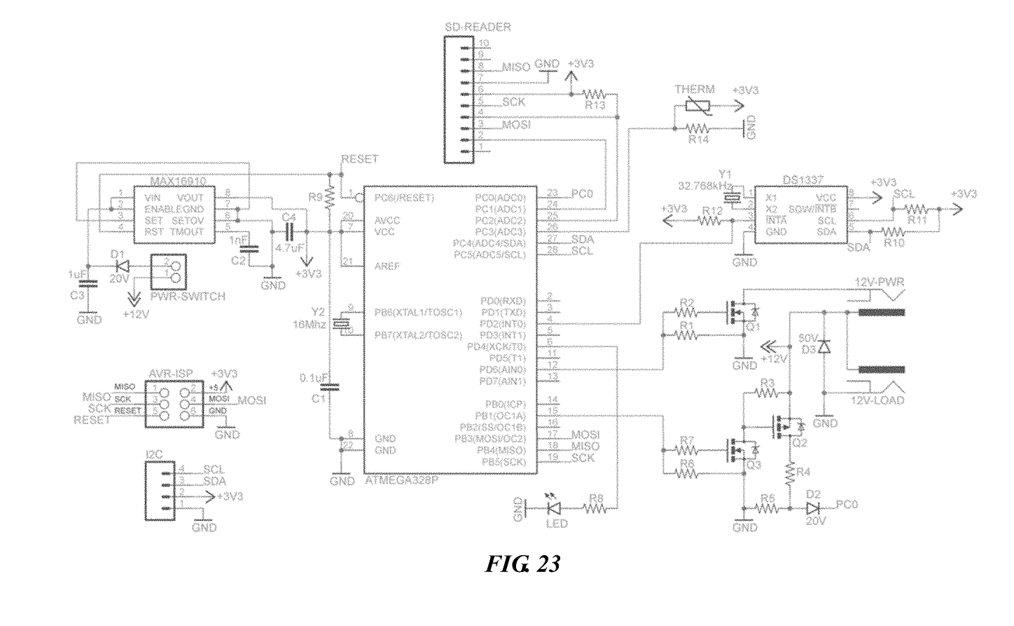

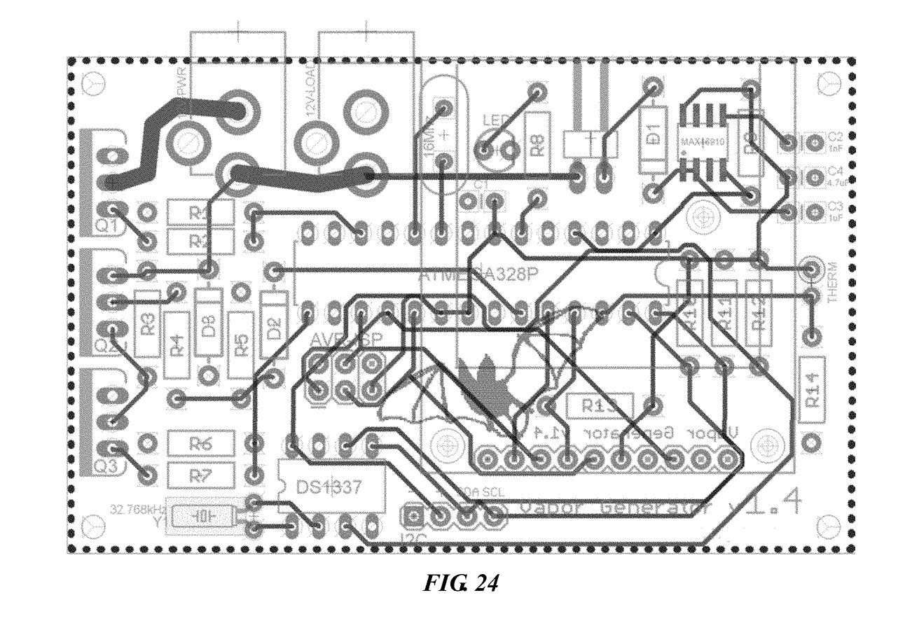

A circuit board revision of the controller is rendered in a schematic view (FIG. 23) and the controller and programmer in a PCB view (FIG. 24).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1-19. (canceled)

20. The method of claim 19, wherein the animal is a bat, wherein the microbial infection comprises Pseudogymnoascus destructans.

21. A method of automatically dispersing a liquid in aerosol form comprising:
receiving, by a computing device, one or more input parameters;
determining, by the computing device based on the input parameters, how long to run a nebulizer unit operably connected with the computing device to achieve a concentration of a compound in an airspace; and
running the nebulizer unit for the determined run time.

22. The method of claim 21, wherein the compound to be dispersed in aerosol form comprises essential oils, VOCs or VOC formulations.

23. The method of claim 21, wherein the input parameters include one or more of an off time for the nebulizer unit, a run time for the nebulizer unit, a start delay for the nebulizer unit, a volume of airspace where the nebulizer unit is to be used, an air turnover rate in the airspace, a barometric pressure in the airspace, a molecular weight of the compound in the reservoir, a desired concentration of the compound in the airspace, and how often to raise the airspace to the desired concentration.

24. The method of claim 21, wherein the determined run time is determined using at least in part an ideal gas law.

25. The method of claim 21, wherein the computing device executes computer-readable instructions to delay a start of the nebulizer unit; turn on the nebulizer unit for a time (T=Run Time); apply a conversion factor to the run time, T, to determine TConv, where the conversion factor is based on a temperature and voltage of a power source that provides power to the nebulizer unit; and, if TConv is less than run time T, then the nebulizer unit is turned on for an additional time period as determined by T−Tconv.

26. The method of claim 21, wherein the AAU is use to treating or preventing fungal infection in an animal.

27. The method of claim 26, wherein the animal is a bat, wherein the fungus comprises Pseudogymnoascus destructans.

28. A method for treating or preventing fungal infection in an animal, comprising exposing the animal to a composition comprising one or more volatile organic compounds (VOCs) in a quantity sufficient to inhibit or reduce fungal growth in the animal.

29. The method of claim 28, wherein the VOC is selected from the group consisting of 2-ethyl-1-hexanol, benzaldehyde, benzothiazole, decanal, nonanal, and N,N-dimethyloctylamine.

30. The method of claim 29, wherein the composition comprises 2-ethyl-1-hexanol and benzaldehyde; 2-ethyl-1-hexanol and nonanal; 2-ethyl-1-hexanol and decanal; or 2-ethyl-1-hexanol and N,N-dimethyloctylamine.

31. The method of claim 29, wherein the composition comprises 2-ethyl-1-hexanol, benzaldehyde, and decanal.

32. The method of claim 29, wherein the composition comprises 2-ethyl-1-hexanol, nonanal, and decanal.

33. The method of claim 28, wherein the VOC is selected from the group consisting of propionoic acid, 2-nonanone, undecene, styrene, β-phenylethanol, and dimethyl sulfide.

34. The method of claim 28, wherein the animal is a bat, wherein the fungus comprises Pseudogymnoascus destructans.
